(19) United States
(12) Patent Application Publication
(10) Pub. No.: US 2024/0175083 A1
(43) Pub. Date: May 30, 2024

(54) METHODS AND COMPOSITIONS FOR ANALYTE DETECTION WITH CONTROLLED POLYMERIZATION REACTIONS

(71) Applicant: 10x Genomics, Inc., Pleasanton, CA (US)

(72) Inventors: Felice Alessio BAVA, Rome (IT); Peter SMIBERT, Danville, CA (US)

(21) Appl. No.: 18/501,746

(22) Filed: Nov. 3, 2023

Related U.S. Application Data
(60) Provisional application No. 63/422,822, filed on Nov. 4, 2022.

(51) Int. Cl. C12Q 1/6841; C12Q 1/682

(52) U.S. Cl. CPC C12Q 1/6841; C12Q 1/682

(57) ABSTRACT

In some aspects, the present disclosure relates to methods, compositions and kits for analyzing a biological sample using a hybridization-based polymerization reaction (HPR). In some aspects, a target nucleic acid molecule is detected via an HPR such as hybridization chain reaction (HCR). In some aspects, the HPR is initiated and terminated by an initiator sequence and terminator sequence, respectively, and the initiator sequence and terminator sequence are associated with the same target nucleic acid molecule.

### CROSS-REFERENCE TO RELATED APPLICATION

This application claims priority to U.S. Provisional Patent Application No. 63/422,822, filed Nov. 14, 2022, entitled “METHODS AND COMPOSITIONS FOR ANALYTE DETECTION WITH CONTROLLED POLYMERIZATION REACTIONS,” which is herein incorporated by reference in its entirety for all purposes.

### FIELD

The present disclosure generally relates to methods and compositions for detection of a target analyte in a sample.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, in situ assays are important tools, for example, for understanding the molecular basis of cell identity and developing treatment for diseases. Certain existing in situ analyte detection methods can yield detection signals outside of an optimal range. For example, signals with small size and/or weak intensity may not reach a threshold of detection. Alternatively, signals with large size and/or high intensity may result in optical overcrowding, especially when in close proximity. In either case, sensitivity can be compromised, reducing the quality of image analysis. There is a need for new and improved methods for in situ assays. The present disclosure addresses these and other needs.

### BRIEF SUMMARY

In some embodiments, provided herein is a method for analyzing a biological sample, comprising: (a) contacting the biological sample with a plurality of hybridization-based polymerization reaction (HPR) monomers comprising a first and a second HPR monomer species, wherein hybridization of a monomer of the first HPR monomer species to an initiator sequence initiates an HPR reaction to generate an amplification product (e.g., a polymeric HPR product), wherein: the first HPR monomer species and second HPR monomer species are each capable of hybridizing to another HPR monomer species; at least one of the HPR monomer species is capable of hybridizing to a terminator sequence; and the initiator sequence and terminator sequence are each independently comprised by (i) a target nucleic acid molecule, or (ii) an adapter probe hybridized directly or indirectly to the target nucleic acid molecule; and (b) detecting the polymeric HPR product, thereby detecting the target nucleic acid molecule.

In any of the embodiments herein, the HPR can be a hybridization chain reaction (HCR), the plurality of HPR monomers can be a plurality of HCR monomers, the first HPR monomer species can be a first HCR monomer species, the second HPR monomer species can be a second HCR monomer species, and the HPR product can be an HCR product. In any of the embodiments herein, the HCR monomers can be provided as DNA hairpins. In some of any of the embodiments herein, the HCR monomers provided as DNA hairpins do not form a polymeric HPR product in the absence of the initiator. In any of the embodiments herein, the plurality of HCR monomers can consist of a first and second HCR monomer species; the first HCR monomer species can comprise a portion A1 and a portion A2; the second HCR monomer species can comprise a portion B1 and a portion B2; A1 can be capable of hybridizing to the initiator; A1 can be capable of hybridizing to B2; A2 can be capable of hybridizing to B1; and A2 and/or B2 can be capable of hybridizing to the terminator sequence.

In any of the embodiments herein, a portion of an HPR monomer of the polymeric HPR product can hybridize to the terminator sequence. In any of the embodiments herein, hybridization of the portion of the HPR monomer of the polymeric HPR product to the terminator sequence can inhibit the portion of the HPR monomer from hybridizing to another HPR monomer. In any of the embodiments herein, the portion of the HPR monomer of the polymeric HPR product hybridized to the terminator sequence can have a higher melting temperature than the portion of the HPR monomer of the polymeric HPR product hybridized to another HPR monomer. In any of the embodiments herein, the portion of the HPR monomer of the polymeric HPR product can have greater complementarity to the terminator sequence than to another HPR monomer. In any of the embodiments herein, hybridization of the portion of the HPR monomer of the polymeric HPR product to the terminator sequence can terminate the HPR. In any of the embodiments herein, the terminator sequence can comprise deoxyribonucleic acid (DNA), ribonucleic acid (RNA) and/or locked nucleic acid (LNA).

In any of the embodiments herein, the terminator sequence can be comprised by the target nucleic acid molecule. In any of the embodiments herein, the terminator sequence can be comprised by an adapter probe further comprising a hybridization region that hybridizes directly to the target nucleic acid molecule or to a branch probe or branch probe complex that hybridizes to the target nucleic acid molecule. In any of the embodiments herein, the terminator sequence can be on a 5′ and/or 3′ overhang of the adapter probe comprising the terminator sequence, wherein the overhang does not directly hybridize to the target nucleic acid molecule or to a branch probe or branch probe complex. In any of the embodiments herein, the adapter probe comprising the terminator sequence can comprise multiple copies of the terminator sequence.

In any of the embodiments herein, the initiator sequence can be comprised by an adapter probe further comprising a hybridization region that hybridizes directly to the target nucleic acid molecule or to a branch probe or branch probe complex that hybridizes to the target nucleic acid molecule. In any of the embodiments herein, the initiator sequence can be on a 5′ and/or 3′ overhang of the adapter probe comprising the initiator sequence, wherein the overhang does not directly hybridize to the target nucleic acid molecule or to a branch probe or branch probe complex. In any of the embodiments herein, the adapter probe comprising the initiator sequence can comprise two or more separate nucleic acid molecules.

In any of the embodiments herein, the adapter probe comprising the initiator sequence can be a split adapter probe, wherein: the split adapter probe comprises a first adapter oligonucleotide comprising a first portion of the initiator sequence and a second adapter oligonucleotide comprising a second portion of the initiator sequence; each adapter oligonucleotide further comprises a hybridization region, and the hybridization regions of the first and second adapter oligonucleotides hybridize to adjacent sequences on the target nucleic acid molecule, or to sequences separated by fewer than about 100 intervening nucleotides on the target nucleic acid molecule; and upon hybridization of the first and second adapter oligonucleotides to the target nucleic acid molecule, the first and second portion of the initiator sequence are brought into proximity and hybridize to a monomer of the first HPR monomer species, thereby initiating an HPR reaction to generate a polymeric HPR product.

In any of the embodiments herein, the initiator sequence and terminator sequence can be comprised by the same adapter probe. In any of the embodiments herein, the initiator sequence and terminator sequence can be comprised by different adapter probes.

In any of the embodiments herein, a plurality of adapter probes comprising the terminator sequence can hybridize to the target nucleic acid or to a branch probe or branch probe complex that is hybridized to the target nucleic acid. In any of the embodiments herein, at least 2, at least 5, at least 10, at least 15, or at least 20 adapter probes comprising the terminator sequence can hybridize to a branch probe or branch probe complex that is hybridized to the target nucleic acid. In any of the embodiments herein, a plurality of branch probes or branch probe complexes can each be hybridized to (i) a plurality of adapter probes comprising the terminator sequence, and (ii) the target nucleic acid molecule. In any of the embodiments herein, the branch probe complex can be a branched hybridization complex.

In any of the embodiments herein, the method can comprise contacting the biological sample with (i) the plurality of HPR monomers, (ii) one or more adapter probes, each independently comprising the initiator sequence and/or terminator sequence, and (iii) the branch probe or branch probe complexes, simultaneously or sequentially in any order. In any of the embodiments herein, the branch probe and/or branch probe complex can be hybridized to the target nucleic acid molecule prior to contacting the sample with the plurality of HPR monomers and/or one or more adapter probes. In any of the embodiments herein, the adapter probe comprising the terminator sequence can be hybridized to the target nucleic acid or branch probe and/or branch probe complex prior to contacting the sample with the plurality of HPR monomers. In any of the embodiments herein, the adapter probe comprising the terminator sequence can be hybridized to the target nucleic acid or branch probe and/or branch probe complex subsequent to contacting the sample with the plurality of HPR monomers.

In any of the embodiments herein, the method can further comprise contacting the sample with a terminator oligonucleotide that comprises the terminator sequence and does not comprise a separate sequence for hybridization directly or indirectly to the target nucleic acid molecule.

In any of the embodiments herein, the polymeric HPR product can comprise fewer HPR monomers than a polymeric HPR product generated in a comparable HPR reaction carried out in the absence of the terminator sequence.

In any of the embodiments herein, at least a fraction of the HPR monomers can be labeled with a detectable label. In any of the embodiments herein, the detectable label can be a fluorescent label. In any of the embodiments herein, detecting the polymeric HPR product can comprise detecting a signal generated from the detectable label. In any of the embodiments herein, the signal generated from the detectable label can have a reduced size and/or intensity in comparison to a signal generated from the detectable label in a comparable HPR reaction carried out in the absence of the terminator sequence.

In any of the embodiments herein, the target nucleic acid molecule can be detected in situ in the biological sample. In any of the embodiments herein, the target nucleic acid molecule can be DNA. In any of the embodiments herein, the target nucleic acid molecule can be RNA.

In any of the embodiments herein, the biological sample can be non-homogenized. In any of the embodiments herein, the biological sample can be selected from the group consisting of a formalin-fixed, paraffin-embedded (FFPE) sample, a frozen tissue sample, and a fresh tissue sample. In any of the embodiments herein, the biological sample can be fixed. In any of the embodiments herein, the biological sample can be not fixed. In any of the embodiments herein, the biological sample can be permeabilized. In any of the embodiments herein, the biological sample can be embedded in a matrix. In any of the embodiments herein, the matrix comprises a hydrogel. In any of the embodiments herein, the biological sample can be cleared. In any of the embodiments herein, the clearing comprises contacting the biological sample with a proteinase. In any of the embodiments herein, the biological sample can be cross-linked. In any of the embodiments herein, the biological sample can be a tissue slice between about 1 μm and about 50 μm in thickness. In any of the embodiments herein, the tissue slice is between about 5 μm and about 35 μm in thickness.

In some embodiments, provided herein is a composition, comprising a plurality of HPR monomers comprising a first HPR monomer species and a second HPR monomer species, wherein: the plurality of HPR monomers are configured to (a) polymerize to generate a polymeric HPR product when a monomer of the first HPR monomer species hybridizes to an initiator sequence, and (b) terminate polymerization when a monomer of the HPR product hybridizes to a terminator sequence; the first HPR monomer species and second HPR monomer species are each capable of hybridizing to another HPR monomer species; and the initiator sequence and terminator sequence are each independently comprised by (i) a target nucleic acid molecule, or (ii) an adapter probe hybridized directly or indirectly to the target nucleic acid molecule. In any of the embodiments herein, the composition can further comprise an adapter probe comprising the initiator sequence, and/or an adapter probe comprising the terminator sequence. In any of the embodiments herein, the composition can further comprise an adapter probe comprising the initiator sequence and the terminator sequence. In any of the embodiments herein, the composition can further comprise one or more of a branch probe or a branch probe complex, wherein each branch probe or branch probe complex is capable of hybridizing to (a) the target nucleic acid molecule, and (b) an adapter probe comprising the initiator and/or terminator sequence. In any of the embodiments herein, the composition can further comprise the target nucleic acid. In some embodiments, provided herein is a kit comprising any of the compositions provided herein.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1D show schematics illustrating exemplary hybridization-based polymerization reactions (HRPs) with or without a terminator sequence.

FIGS. 2A-2F show schematics illustrating exemplary configurations of a target nucleic acid, an initiator, and a terminator sequence for HPR. HPR monomers and polymeric HPR product are not shown.

FIGS. 3A-3E show schematics illustrating exemplary configurations of a target nucleic acid, an initiator sequence, and a terminator sequence for HPR, in which multiple copies of the terminator sequence are provided. HPR monomers and polymeric HPR product are not shown.

FIG. 4 shows schematics illustrating an exemplary hybridization chain reaction (HCR) comprising a first and second HCR monomer provided as DNA hairpins, in which the reaction is terminated by a terminator sequence.

### DETAILED DESCRIPTION

All publications, comprising patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. OVERVIEW

Hybridization-based polymerization reactions (HPRs), such as hybridization chain reaction (HCR), provide useful methods for detecting target analytes, such as nucleic acids, in a sample. In HPRs, two or more nucleic acid monomer species undergo a hybridization-based polymerization reaction upon hybridization of one of the monomers to an initiator sequence, which may be present in a target analyte. In some aspects, an HPR is a non-enzymatic amplification reaction wherein two or more nucleic acid monomer species become incorporated in a generated amplification product (e.g., a polymeric HPR product). The resulting polymerized HPR product can be detected, thus revealing the presence of the target analyte in a sample.

HCR is a commonly utilized method of HPR for detection of target analytes (Dirks and Pierce, PNAS 101(43): 15275-15278 (2004); Choi et al., Development 145(12): dev165753 (2018)). In HCR, typically a first and second monomer species are provided as kinetically trapped DNA hairpins which do not polymerize in the absence of an initiator. Upon hybridization of a monomer of the first species to an initiator sequence, the hairpin of the monomer of the first species is opened, exposing a sequence that hybridizes to a monomer of the second species. The hairpin of the monomer of the second species in turn is opened and hybridizes to another monomer of the first species. The HCR proceeds as a growing chain of hybridized alternating monomers in a nicked double helix formation.

A challenge in using HPR methods such as HCR to detect analytes is that polymerization is not well-controlled (Figg et al., J. Am. Chem. Soc. 142(19):8596-8601 (2020)). In conventional HCR, for example, once the reaction is initiated, polymerization may continue for as long as additional hairpin monomers are present in the sample. Consequently, HCR products may be larger than desired (i.e. comprise a large number of monomers), and/or HCR products resulting from similar starting conditions may vary widely in size (i.e. have high dispersity). Because the signal detected from an HPR product is typically proportional to the degree of polymerization, this can result in signals being undesirably large and/or intense, and/or the signals generated from one or more target analytes being highly variable, thereby compromising detection and quantification of analytes. Thus, there is a need for methods for controlling polymerization in HPR in order to optimize detection and quantification.

In some aspects, provided herein are methods and compositions for controlling HPRs using a terminator sequence that is directly or indirectly associated with the target nucleic acid. The terminator sequence is configured such that at least one monomer species of the HPR is capable of hybridizing to the terminator sequence, and when a monomer of the growing polymeric HPR product hybridizes to the terminator sequence, the monomer is inhibited from hybridizing to another monomer, such that the polymerization reaction is terminated. Controlled termination of HPR by these methods can improve signal detection by reducing HPR product size and/or variability.

An additional advantage of the compositions and methods described herein is that they are flexible, and may be configured or empirically determined by a user to achieve the desired control over HPR product polymerization. For example, the HPR can be designed such that the terminator sequence is comprised by a target nucleic acid, or comprised by a probe that hybridizes directly or indirectly to the target nucleic acid. The proximity and abundance of the one or more terminator sequences to the initiator sequence can also be readily determined by the user. A configuration with multiple terminator sequences placed in close proximity to the initiator sequence may be determined by a user to limit polymerization. In some embodiments, provided herein are methods for performing a controlled amplification reaction to generate an amplification product (e.g., a polymeric HPR product) using the terminator sequence.

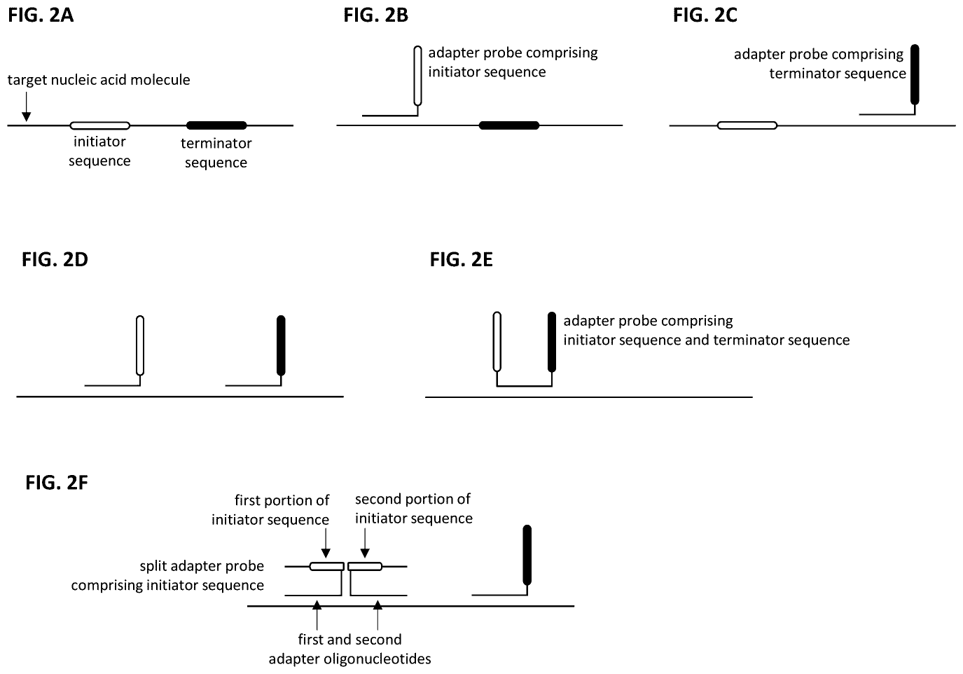

In some aspects, provided herein is a method for analyzing a biological sample. In some aspects, the method comprises contacting the biological sample with a plurality of hybridization-based polymerization reaction (HPR) monomers. In some embodiments, the HPR monomers comprise a first HPR monomer species and a second HPR monomer species. In some embodiments, hybridization of a monomer of the first HPR monomer species to an initiator sequence initiates an HPR reaction to generate a polymeric HPR product. In some embodiments, the first HPR monomer species and second HPR monomer species are each capable of hybridizing to another HPR monomer species. In some embodiments, at least one of the HPR monomer species is capable of hybridizing to a terminator sequence. In some embodiments, the initiator sequence and the terminator sequence are each independently comprised by (i) a target nucleic acid molecule, and/or (ii) an adapter probe bound directly or indirectly to the target nucleic acid molecule (see, e.g., FIGS. 2A-2F). In some embodiments, the method further comprises detecting the polymeric HPR product, thereby detecting the target nucleic acid molecule.

In some aspects of the method, the HPR is a hybridization chain reaction (HCR). In some embodiments, the plurality of HPR monomers is a plurality of HCR monomers. In some embodiments, the first HPR monomer species is a first HCR monomer species. In some embodiments, the second HPR monomer species is a second HCR monomer species. In some embodiments, the HPR product is an HCR product. In some embodiments, the HCR monomers are provided as DNA hairpins.

In some embodiments, the HCR monomers provided as DNA hairpins do not form an amplification product (e.g., a polymeric HPR product) in the absence of the initiator. In some embodiments, the plurality of HCR monomers consist of a first and second HCR monomer species. In some embodiments, the first HCR monomer species comprises a portion A1 and a portion A2. In some embodiments, the portion A1 comprises an input domain and the portion A2 comprises an output domain, as described herein. In some embodiments, the second HCR monomer species comprises a portion BI and a portion B2. In some embodiments, the portion BI comprises an input domain and the portion B2 comprises an output domain, as described herein. In some embodiments, A1 is capable of hybridizing to the initiator. In some embodiments, A1 is capable of hybridizing to B2. In some embodiments, A2 is capable of hybridizing to B1. In some embodiments, A2 and/or B2 is capable of hybridizing to the terminator sequence. In some embodiments, A2 and B2 are capable of hybridizing to different terminator sequences. In some embodiments, A2 and B2 are capable of hybridizing to the same terminator sequence.

In some embodiments, a portion of an HPR monomer of the polymeric HPR product hybridizes to the terminator sequence. In some embodiments, hybridization of the portion of the HPR monomer of the polymeric HPR product to the terminator sequence inhibits the portion of the HPR monomer from hybridizing to another HPR monomer. In some embodiments, the portion of the HPR monomer of the polymeric HPR product hybridized to the terminator sequence has a higher melting temperature than the portion of the HPR monomer of the polymeric HPR product hybridized to another HPR monomer. In some embodiments, the portion of the HPR monomer of the polymeric HPR product has greater complementarity to the terminator sequence than to another HPR monomer. In some embodiments, hybridization of the portion of the HPR monomer of the polymeric HPR product to the terminator sequence terminates the HPR. In some embodiments, the terminator sequence comprises deoxyribonucleic acid (DNA), ribonucleic acid (RNA) and/or locked nucleic acid (LNA). In some embodiments, the terminator sequence is comprised by the target nucleic acid molecule. In some embodiments, the terminator sequence is comprised by an adapter probe further comprising a hybridization region. In some embodiments, the hybridization region hybridizes directly to the target nucleic acid molecule. In some embodiments, the hybridization region hybridizes to a branch probe or branch probe complex that hybridizes to the target nucleic acid molecule. In some embodiments, the terminator sequence is on a 5′ and/or 3′ overhang of the adapter probe comprising the terminator sequence, wherein the overhang does not directly hybridize to the target nucleic acid molecule or to a branch probe or branch probe complex. In some embodiments, the adapter probe comprising the terminator sequence comprises multiple copies of the terminator sequence.

In some embodiments, the initiator sequence is comprised by an adapter probe further comprising a hybridization region that hybridizes directly to the target nucleic acid molecule or to a branch probe or branch probe complex that hybridizes to the target nucleic acid molecule. In some embodiments, the initiator sequence is on a 5′ and/or 3′ overhang of the adapter probe comprising the initiator sequence. In some embodiments, the overhang does not directly hybridize to the target nucleic acid molecule or to a branch probe or branch probe complex. In some embodiments, the adapter probe comprising the initiator sequence comprises two or more separate nucleic acid molecules. In some embodiments, the adapter probe comprising the initiator sequence is a split adapter probe. In some embodiments, the split adapter probe comprises a first adapter oligonucleotide comprising a first portion of the initiator sequence and a second adapter oligonucleotide comprising a second portion of the initiator sequence. In some embodiments, each adapter oligonucleotide further comprises a hybridization region. In some embodiments, the hybridization regions of the first and second adapter oligonucleotides hybridize to adjacent sequences on the target nucleic acid molecule. In some embodiments, the hybridization regions of the first and second adapter oligonucleotides hybridize to sequences separated by fewer than about 100 intervening nucleotides on the target nucleic acid molecule. In some embodiments, upon hybridization of the first and second adapter oligonucleotides to the target nucleic acid molecule, the first and second portion of the initiator sequence are brought into proximity and hybridize to a monomer of the first HPR monomer species, thereby initiating an HPR reaction to generate a polymeric HPR product.

In some embodiments, the initiator sequence and terminator sequence are comprised by the same adapter probe. In some embodiments, the initiator sequence and terminator sequence are comprised by different adapter probes.

In some embodiments, a plurality of adapter probes comprising the terminator sequence hybridize to the target nucleic acid or to a branch probe or branch probe complex that is hybridized to the target nucleic acid. In some embodiments, at least 2, at least 5, at least 10, at least 15, or at least 20 adapter probes comprising the terminator sequence hybridize to a branch probe or branch probe complex that is hybridized to the target nucleic acid. In some embodiments, a plurality of branch probes or branch probe complexes are each hybridized to (i) a plurality of adapter probes comprising the terminator sequence, and (ii) the target nucleic acid molecule. In some embodiments, the branch probe complex is a branched hybridization complex.

In some embodiments, the method comprises contacting the biological sample with (i) the plurality of HPR monomers, (ii) one or more adapter probes, each independently comprising the initiator sequence and/or terminator sequence, and (iii) the branch probe or branch probe complex, simultaneously or sequentially in any order. In some embodiments, the branch probe and/or branch probe complex is hybridized to the target nucleic acid molecule prior to contacting the sample with the plurality of HPR monomers and/or one or more adapter probes. In some embodiments, the adapter probe comprising the terminator sequence is hybridized to the target nucleic acid or branch probe and/or branch probe complex prior to contacting the sample with the plurality of HPR monomers. In some embodiments, the adapter probe comprising the terminator sequence is hybridized to the target nucleic acid or branch probe and/or branch probe complex subsequent to contacting the sample with the plurality of HPR monomers.

In some embodiments, the method further comprises contacting the sample with a terminator oligonucleotide. In some embodiments, the terminator oligonucleotide comprises the terminator sequence and does not comprise a separate sequence for hybridization directly or indirectly to the target nucleic acid molecule.

In some embodiments, the polymeric HPR product comprises fewer HPR monomers than a polymeric HPR product generated in a comparable HPR reaction carried out in the absence of the terminator sequence.

In some embodiments, at least a fraction of the HPR monomers are labeled with a detectable label. In some embodiments, the detectable label is a fluorescent label. In some embodiments, detecting the polymeric HPR product comprises detecting a signal generated from the detectable label. In some embodiments, the signal generated from the detectable label has a reduced size and/or intensity in comparison to a signal generated from the detectable label in a comparable HPR reaction carried out in the absence of the terminator sequence.

In some embodiments, the target nucleic acid molecule is detected in situ in the biological sample. In some embodiments, the target nucleic acid molecule is DNA. In some embodiments, the target nucleic acid molecule is RNA.

In some embodiments, the biological sample is non-homogenized. In some embodiments, the non-homogenized biological sample is selected from the group consisting of a formalin-fixed, paraffin-embedded (FFPE) sample, a frozen tissue sample, and a fresh tissue sample. In some embodiments, the biological sample is fixed. In some embodiments, the biological sample is a cell or tissue sample. In some embodiments, the biological sample is not fixed. In some embodiments, the biological sample is permeabilized. In some embodiments, the biological sample is embedded in a matrix. In some embodiments, the matrix comprises a hydrogel. In some embodiments, the biological sample is cleared. In some embodiments, the clearing comprises contacting the biological sample with a proteinase. In some embodiments, the biological sample is cross-linked. In some embodiments, the biological sample is a tissue slice between about 1 μm and about 50 μm in thickness. In some embodiments, the tissue slice is between about 5 μm and about 35 μm in thickness.

In some embodiments, provided herein is a composition, comprising a plurality of HPR monomers comprising a first HPR monomer species and a second HPR monomer species. In some embodiments, the plurality of HPR monomers are configured to (a) polymerize to generate a polymeric HPR product when a monomer of the first HPR monomer species hybridizes to an initiator sequence, and (b) terminate polymerization when a monomer of the HPR product hybridizes to a terminator sequence. In some embodiments, the first HPR monomer species and second HPR monomer species are each capable of hybridizing to another HPR monomer species. In some embodiments, the initiator sequence and terminator sequence are each independently comprised by (i) a target nucleic acid molecule, or (ii) an adapter probe hybridized directly or indirectly to the target nucleic acid molecule. In some embodiments, the composition further comprises an adapter probe comprising the initiator sequence, and/or an adapter probe comprising the terminator sequence. In some embodiments, the composition further comprises an adapter probe comprising the initiator sequence and the terminator sequence. In some embodiments, the composition further comprises one or more of a branch probe or a branch probe complex. In some embodiments, each branch probe or branch probe complex is capable of hybridizing to (a) the target nucleic acid molecule, and (b) an adapter probe comprising the initiator and/or terminator sequence. In some embodiments, the composition further comprises the target nucleic acid. In some embodiments, provided herein is a kit comprising the composition.

### II. SAMPLES, ANALYTES, AND TARGET SEQUENCES

### A. Samples and Sample Processing

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, a cell pellet, a cell block, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products or polymeric HPR products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample is fixed. In some embodiments, the biological sample is not fixed.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes. In some embodiments, one or more post-fixing step is performed after a hybridization complex comprising a probe and a target is formed in a sample. In some embodiments, one or more post-fixing step is performed prior to a ligation reaction disclosed herein.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed, e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). In some aspects, the embedding material can be applied to the sample one or more times. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, US2016/0024555, US2019/0276881, US2020/0071751, US2021/0292834, US2021/0230692, and US2021/0310052, the entire contents of which are incorporated herein by reference. A method disclosed herein may comprise embedding tissue or cell samples within conductive hydrogels. U.S. Pat. Publ. No. 2011/0256183 (Frank et al.), U.S. Pat. No. 10,138,509 (Church et al.), U.S. Pat. No. 10,545,075 (Deisseroth et al.) and U.S. Pat. Publ. No. 2019/0233878 (Delaney et al.), which are herein incorporated by reference, describe hydrogels and their use for embedding tissues and cells.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of each of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly or irreversibly cross-linked prior to, during, or after an assay step disclosed herein. A cross-linking agent includes a chemical agent, or even light, that facilitates the attachment of one molecule to another molecule. Cross-linking agents can be protein-nucleic acid cross-linking agents, nucleic acid-nucleic acid cross-linking agents, and/or protein-protein cross-linking agents. In some embodiments, a cross-linking agent is a reversible cross-linking agent. In some embodiments, a cross-linking agent is a non-reversible cross-linking agent.

In some embodiments, the sample to be analyzed is contacted with a protein-nucleic acid cross-linking agent, a nucleic acid-nucleic acid cross-linking agent, a protein-protein cross-linking agent or any combination thereof. In some examples, a cross-linker is a reversible cross-linker, such that the cross-linked molecules can be easily separated. In some examples, a cross-linker is a non-reversible cross-linker, such that the cross-linked molecules cannot be easily separated. In some examples, a cross-linker is light, such as UV light. In some examples, a cross linker is light activated. These cross-linkers include formaldehyde, disuccinimidyl glutarate, UV-254, psoralens and their derivatives such as aminomethyltrioxsalen, glutaraldehyde, ethylene glycol bis[succinimidylsuccinate], and other compounds, including those described in the Thermo Scientific Pierce Cross-linking Technical Handbook, Thermo Scientific (2009) as available on the world wide web at piercenet.com/files/1601673_Cross-link_HB_Intl.pdf.

In some embodiments, target molecules can be present within a three dimensional matrix material and covalently attached to the three dimensional matrix material such that the relative position of each target molecule is fixed, e.g., immobilized, within the three dimensional matrix material. In this manner, a three-dimensional matrix of covalently bound target molecules of any desired sequence is provided. Each target molecule has its own three dimensional coordinates within the matrix material and each target molecule represents information. In this manner, a large amount of information can be stored in a three dimensional matrix.

In some embodiments, a cross-linkable probe is used to anchor target molecules to a three dimensional matrix such that the relative position of each target molecule is fixed. In embodiments, the sample is contacted with a poly-dT anchor probe to bind and anchor polyadenylated (polyA) RNAs to the matrix. In some embodiments, the anchor probe (e.g., the poly-dT anchor probe) comprises a terminal acrydite moiety or other crosslinkable moiety, which can be covalently incorporated into the matrix (e.g., during matrix polymerization). In some embodiments, the poly-dT anchor probe can be about 10 to 20 nucleotides in length (e.g., about 15-nucleotides in length). In some embodiments, the anchor probe can comprise locked-nucleic acid bases to stabilize the hybridization of the poly-dT anchor probe to poly A tails of the RNAs.

According to a further aspect, the target molecules can be amplified products of an analyte, such as amplicons or amplification products (e.g., polymeric HPR products described herein) produced within the three dimensional matrix material. The amplicons or amplification products can then be covalently attached to the matrix, for example, by copolymerization or cross-linking. This results in a structurally stable and chemically stable three dimensional matrix of target molecules. According to this aspect, the three dimensional matrix of target molecules allows for prolonged information storage and read-out cycles. Furthermore, the position of the target molecules in the sample can be stable.

In some aspects, the analytes, polynucleotides and/or amplification product (e.g., amplicon or polymeric HPR product) of an analyte or a probe bound thereto can be anchored to a polymer matrix. For example, the polymer matrix can be a hydrogel. In some embodiments, the polymer matrix comprises functional moieties. In some embodiments, one or more of the polynucleotide probe(s) and/or amplification product (e.g., amplicon or polymeric HPR product) thereof can be modified to contain functional groups that can be used as an anchoring site to attach the polynucleotide probes and/or amplification product to a polymer matrix. In some embodiments, crosslinking chemistry may be used to anchor functional moieties of the one or more of the polynucleotide probe(s) and/or amplification product (e.g., amplicon or polymeric HPR products) to other molecules and/or to the polymer matrix. For example, any suitable functional moieties can be used, such as an amine, acrydite, alkyne, biotin, azide, and thiol. In some embodiments for crosslinking, the functional moiety may be cross-linked to modified dNTP or dUTP or both. In some cases, a combination of anchoring approaches (e.g., functional moieties) can be used, e.g., to anchor one or more types of molecules to the polymer matrix.

In some embodiments, the anchoring may comprise using an acrylamide group or click chemistry moiety. In some aspects, one or more of the polynucleotide probe(s) and/or amplification product (e.g., amplicon or polymeric HPR products) thereof can comprise modified nucleotides that may have the functional group directly (e.g., acrylamide, click chemistry) or be further modified (e.g., amine modified with an NHS ester chemistry) to contain the functional group. In some embodiments, click reaction chemistry may be used to couple one or more of the target molecules (or a product or derivative thereof), polynucleotide probe(s), and/or amplification product (e.g., amplicon or polymeric HPR products) to the matrix (e.g., hydrogel). Any suitable click reaction and click reactive groups may be used. In some cases, a molecule may be tethered via a click reaction to a click reactive group functionalized hydrogel matrix (e.g., click gel). For example, the 5′azidomethyl-dUTP can be incorporated into a product or derivative of the target molecule, polynucleotide probe(s), and/or amplification product (e.g., amplicon or polymeric HPR products) and then immobilized to the hydrogel matrix functionalized with alkyne groups. In some embodiments, a buffer can be used for click reaction catalyzation, e.g., a Cu(I)-catalyzed alkyne-azide cycloaddition (abbreviated as CuAAC) click reaction catalyzing buffer, which catalyzes the alkyne-azide bond in the click reaction.

In some embodiments, a product or derivative of the target molecule, polynucleotide probe(s), and/or amplification product (e.g., amplicon or polymeric HPR product) may be functionalized by adding nucleotide triphosphate analogs comprising functional moieties for immobilization. In some examples, the nucleotide triphosphate analogs include, but are not limited to, amino-allyl dUTP, 5-TCO-PEG4-dUTP, C8-Alkyne-dUTP, 5-Azidomethyl-dUTP, 5-Vinyl-dUTP, 5-Ethynyl dUTP, and other nucleotide triphosphate analogs comprising a functional moiety for immobilization by cross-linking, or forming a chemical bond between the molecule and the matrix.

In some embodiments, the matrix comprises a cellular or synthetic matrix that contains chemical moieties (e.g., reactive groups) that can react with the functional moieties in the product or derivative of the target molecule, polynucleotide probe(s), and/or amplification product (e.g., amplicon or polymeric HPR product) through functionalization reactions. For example, amino-allyl dUTP may be cross-linked to endogenous free amine groups present in proteins and other biomolecules present within the endogenous or exogenous cellular matrix, or present in a modified synthetic hydrogel matrix, such as an amine-functionalized polyacrylamide hydrogel formed by copolymerization of polyacrylamide and N-(3-aminopropyl)-methacrylamide. In some cases, nucleoside analogs containing azide functional moieties may be cross-linked to a synthetic hydrogel matrix comprising alkyne functional moieties, such as that formed by copolymerization of acrylamide and propargyl acrylamide.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

(viii) Tissue Permeabilization and Treatment

In some embodiment, the biological sample is permeabilized.

In some embodiments, a biological sample can be permeabilized to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of each of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide (e.g., a target nucleic acid molecule or nucleic acid hybridized directly or indirectly thereto) comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction). In some embodiments, the target nucleic acid molecule is the reporter oligonucleotide or a product thereof. In some embodiments, the target nucleic acid molecule is hybridized directly or indirectly to the reporter oligonucleotide or a product thereof.

A biological sample may comprise one or a plurality of analytes of interest. Methods for performing multiplexed assays to analyze two or more different analytes in a single biological sample are provided.

### B. Analytes

The methods and compositions disclosed herein can be used to detect and analyze a wide variety of different analytes. In some aspects, an analyte can include any biological substance, structure, moiety, or component to be analyzed. In some embodiments, any of the target nucleic acid molecules described herein can correspond to an analyte. For instance, a target nucleic acid molecule can be an endogenous nucleic acid analyte (e.g., DNA or RNA), a product of an endogenous nucleic acid analyte, a probe that directly or indirectly binds to an endogenous nucleic acid analyte, or a product of a probe that directly or indirectly binds to an endogenous nucleic acid analyte. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed.

Analytes of particular interest may include nucleic acid molecules, such as DNA (e.g. genomic DNA, mitochondrial DNA, plastid DNA, viral DNA, etc.) and RNA (e.g. mRNA, microRNA, IRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

In some embodiments, the target nucleic acid molecule is the analyte. In some embodiments, the target nucleic acid molecule is associated with the analyte, such as bound or hybridized directly or indirectly to the analyte or a product thereof. In some embodiments, the target nucleic acid molecule is a circular or circularized probe or probe set, or a product thereof (e.g., an RCA product).

### (i) Endogenous Analytes

In some embodiments, a target molecule herein corresponds to an analyte that is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (TRNA), 5S IRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s IRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence, various probes or probe sets (e.g., HPR monomers described in Section III)), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, a binder or an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 2019/0367969, which are each incorporated by reference herein in their entireties.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### C. Target Sequences and Barcodes

A target sequence for a probe disclosed herein (e.g., an adapter probe comprising an initiator sequence and/or terminator sequence) may be comprised in any analyte disclosed herein, including an endogenous analyte (e.g., a viral or cellular nucleic acid), a labeling agent (e.g., a reporter oligonucleotide attached thereto), or a product of an endogenous analyte and/or a labeling agent.

In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product) or derivative thereof is analyzed. In some embodiments, a labeling agent (or a reporter oligonucleotide attached thereto) that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product) or derivative of a labeling agent that directly or indirectly binds to an analyte in the biological sample is analyzed.

In some embodiments, hybridization comprises the pairing of substantially complementary or complementary nucleic acid sequences within two different molecules, one of which is the endogenous analyte or the labeling agent (e.g., reporter oligonucleotide attached thereto). The other molecule can be another endogenous molecule or another labeling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes or probe sets (e.g., HPR monomers described in Section III) can be hybridized to an endogenous analyte and/or a labeling agent (e.g., a reporter oligonucleotide attached thereto) and each probe may correspond to the target analyte (e.g., target nucleic acid molecule). In some embodiments, the probes or probe sets may comprise one or more barcode sequences. In some embodiments, the nucleic acid probes (e.g., described in Section III) may hybridize to a product of an endogenous analyte and/or a labeling agent that is a ligation product. In some embodiments, a product herein includes a molecule or a complex generated in a series of reactions, e.g., hybridization, ligation, extension, replication, transcription/reverse transcription, and/or amplification, in any suitable combination.

In some embodiments described herein, the analyte comprises or is associated with a target sequence. In some embodiments, a target sequence for a nucleic acid probe described herein is a marker sequence for a given analyte. A marker sequence is a sequence that identifies a given analyte (e.g., alone or in combination with one or more other marker sequences). Thus, in some embodiments, a marker sequence for a given target analyte is specific to that analyte, or unique, such that multiple target analytes can be distinguished from each other.

A “marker sequence” is thus a sequence which marks, is associated with, or identifies a given analyte. It is a sequence by which a given analyte may be detected and distinguished from other analytes. Where an “analyte” comprises a group of related molecules e.g. isoforms or variants or mutants etc., or molecules in a particular class or group, it is not required that a marker is unique or specific to only one particular analyte molecule, and it may be used to denote or identify the analyte as a group. However, where desired, a marker sequence may be unique or specific to a particular specific analyte molecule, e.g. a particular variant. In this way different variants, or isoforms, or mutants may be identified or distinguished from one another.

Where the analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte or labeling agent (e.g., any of products described in Section B (iii) above) as a tag or identifier (ID) sequence (e.g. a barcode) for the analyte or labeling agent. It may thus be a synthetic or artificial sequence.

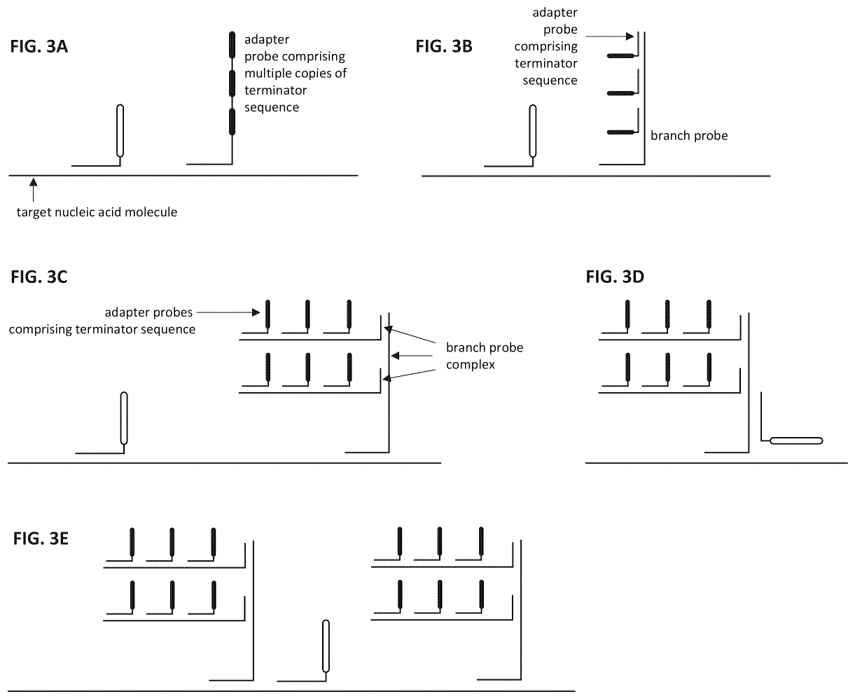

In some embodiments, an endogenous analyte, labeling agent, or a product of an analyte or labeling agent may comprise multiple copies of the target sequence. For example, a probe molecule, or probe component, may comprise multiple copies of a target sequence (e.g., a branch probe or branch probe complex may comprise multiple copies of a target sequence for an adapter probe, as shown in FIGS. 3B-3E). In another example, an amplification product may be generated which comprises multiple copies of the target sequence (e.g., multiple copies of a barcode sequence).

It will be understood that in the case of an analyte, product, or labeling agent comprising multiple target sequences, while each of the target sequences may comprise a binding site for a nucleic acid probe described herein, in practice not all of these binding sites may (or will) be occupied by a nucleic acid probe after nucleic acid probe hybridization. In some embodiments, it suffices that a number, or multiplicity, of such binding sites are bound by a nucleic acid probe. Thus, in some embodiments the nucleic acid probe may hybridize to at least one target sequence present in an analyte, labeling agent, or product of an analyte or labeling agent. In some embodiments, the nucleic acid probe hybridizes to multiple target sequences present in the analyte, labeling agent, or product of an analyte or labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4N complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 2019/0055594 and U.S. Pat. Pub 2021/0164039, which are hereby incorporated by reference in their entireties.

In some embodiments, any analyte can be detected if a target nucleic acid is or can be associated with the analyte. In some embodiments, the analyte is any nucleic acid molecule. In some embodiments, the analyte is a protein. In some embodiments, the analyte consists of at least one of: mRNA, miRNA, lncRNA, rRNA, non-coding RNA, or genomic DNA. In some embodiments, the analyte is comprised of an amino acid sequence. In some embodiments, the analyte is comprised of a complex of molecules. In some embodiments, the analyte is at least one of: DNA, RNA, protein, or small molecule target molecules or complexes in vitro, in situ, or in vivo. In some embodiments, the analyte is a complex of molecules that is made up of at least one of: DNA, RNA, protein, or small molecule target molecules. In some embodiments, the analyte comprises a molecule or complex in vitro, in situ, or in vivo.

### III. NUCLEIC ACID PROBES

Disclosed herein in some aspects are nucleic acid probes and/or probe sets (e.g., polynucleotide probes and/or probe sets) that are introduced into a cell or used to otherwise contact a biological sample such as a tissue sample. The probes (e.g., any HPR monomer, any adapter probe comprising the initiator sequence and/or terminator sequence, any branch probe, or any probe of a branch probe complex) may comprise any of a variety of entities that can hybridize to a nucleic acid, typically by Watson-Crick base pairing, such as DNA, RNA, LNA, PNA, etc. A nucleic acid probe typically contains a targeting sequence that is able to directly or indirectly bind to at least a portion of a target nucleic acid. The nucleic acid probe may be able to bind to a specific target nucleic acid (e.g., an mRNA, or other nucleic acids as discussed herein). In some embodiments, the nucleic acid probes (e.g., the HPR monomers) may be detected using a detectable label, and/or by using secondary nucleic acid probes able to bind to the nucleic acid probes. In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

In some embodiments, provided herein are probes, probe sets, and/or higher order probes. In some embodiments, probes may be hairpin probes or linear probes (e.g., HPR monomers). In some embodiments, a probe can be one that comprises a target recognition sequence and a sequence that does not hybridize to a target nucleic acid (e.g., an adapter probe), such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to the probe itself and/or to one or more other nucleic acids or probes, such as detectably labeled probes. Specific probe designs can vary depending on the application.

### A. HPR Monomers

HPR monomers are nucleic acid molecules (generally oligonucleotides) that are able to assemble to form a polymeric HPR product by hybridization in an HPR reaction initiated by an initiator sequence. In some embodiments, an HPR reaction is propagated by two or more different HPR monomer species. In some embodiments, an HPR reaction is propagated by two HPR monomer species. In some embodiments, each HPR monomer species comprises a plurality of HPR monomers comprising the same sequence.

In some aspects, each HPR monomer comprises an input domain and an output domain. In some aspects, the output domain of an HPR monomer is capable of hybridizing to (e.g., is complementary to) the input domain of another HPR monomer. In some aspects, the input domain of an HPR monomer is also capable of hybridizing to the initiator sequence. In some aspects, the output domain of an HPR monomer is also capable of hybridizing to the terminator sequence.

In some embodiments, the output domain of an individual HPR monomer is not accessible for polymerization unless the input domain of the HPR monomer is hybridized to the initiator sequence or to the output domain of another HPR monomer. In some embodiments, HPR monomers form secondary structures. In some embodiments, hybridization of the initiator sequence or the output domain of a first HPR monomer to the input domain of a second HPR monomer leads to a change in the secondary structure of the second HPR monomer, which allows the output domain of the second HPR monomer to be accessible for polymerization (e.g., hybridization to an input domain of a third HPR monomer). In some embodiments, the output domain of an individual HPR monomer is not accessible for polymerization because it is hybridized to the input domain of the same HPR monomer (i.e. its own input domain). In some embodiments, the HPR monomer forms a double-stranded portion, (e.g., forms a DNA hairpin comprising a stem and loop), resulting from complementarity between the input and output domains. In some embodiments, the 3′ or 5′ end of the input domain comprises a toehold that does not form a portion of the double-stranded portion of the HPR monomer, thereby remaining accessible for hybridization to the initiator sequence or to the output sequence of another HPR monomer. In some embodiments, hybridization of the initiator sequence or output domain of a first HPR monomer to the toehold of a second HPR monomer leads to displacement of the output domain of the second HPR monomer by the initiator sequence or output domain of the first HPR monomer, thereby opening the double-stranded portion (e.g., opening the hairpin) to expose the output domain of the second HPR monomer, making it accessible for polymerization (e.g., for hybridization to the input domain of a third HPR monomer).

In some embodiments, the HPR monomers are hairpin monomers. The hairpin monomers may comprise loops protected by long stems. In other embodiments, monomers with a different secondary structure are provided. The secondary structure is preferably such that the monomers are metastable under reaction conditions in the absence of an initiator nucleic acid. In some embodiments, the HPR monomers are linear monomers.

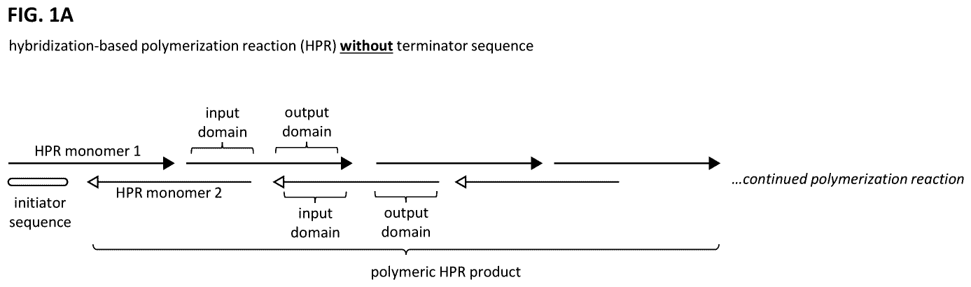

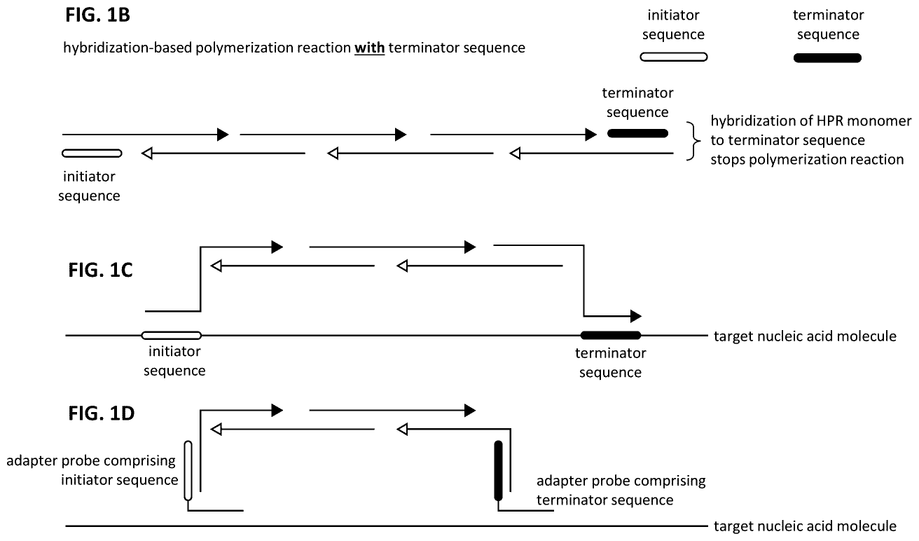

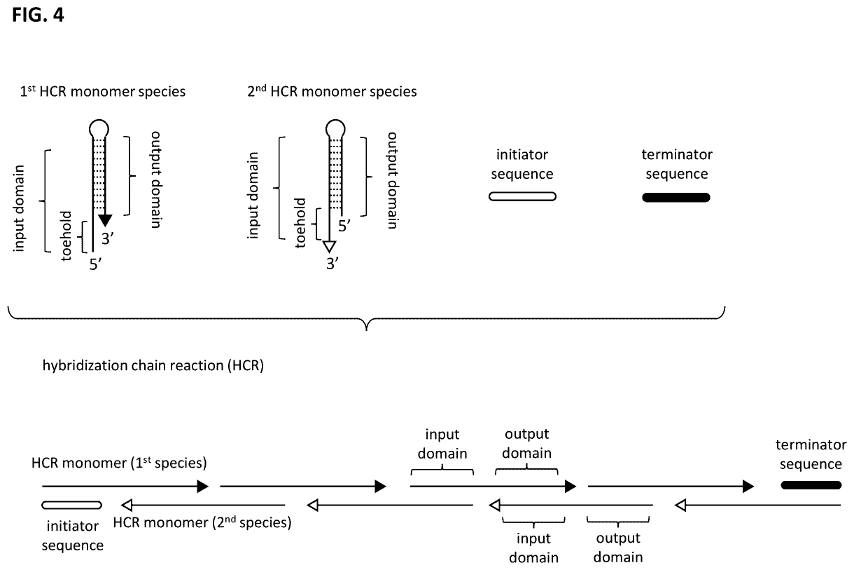

In some embodiments, an HPR reaction is initiated when the initiator sequence hybridizes to the input domain of a first HPR monomer. In some embodiments, hybridization of the initiator sequence to the input domain of the first monomer allows the output domain of the first HPR monomer to hybridize to the input domain of a second HPR monomer, which in turn allows the output domain of the second HPR monomer to hybridize to the input domain of a third monomer, and so on. In some embodiments, the HPR reaction comprises two HPR monomer species that hybridize to the growing amplification product (e.g., polymeric HPR product) in alternating fashion, for example as shown in FIGS. 1A-1D and FIG. 4. In some aspects, an HPR reaction may continue for as long as unincorporated HPR monomers are present. In some embodiments, an HPR reaction is terminated when an HPR monomer of the polymeric HPR product hybridizes to the terminator sequence.

In some embodiments, the HPR reaction is an HCR reaction. In some embodiments, an HCR reaction is propagated by two HCR monomer species which are provided as DNA hairpins. The hairpins may comprise loops protected by long stems. Without being bound by any theory, the secondary structure of hairpins is utilized to increase the energy barrier associated with polymerization. In some embodiments, the HCR monomers comprise an input domain and output domain which form the double-stranded portion of the hairpin as a result of complementarity of the input and output domain. In some embodiments, each HCR monomer comprises a toehold at the 5′ or 3′ end of the input domain. In some embodiments, the HCR monomers do not polymerize in the absence of the initiator sequence. In some embodiments, hybridization of the initiator sequence to the input domain of a monomer of the first HCR monomer species initiates an HCR reaction, wherein HCR monomers of the two HCR monomer species are hybridized to a growing polymeric HCR product in alternating fashion, for example as shown in FIG. 4. HCR reactions are not limited to two HCR monomer species. In some embodiments, an asymmetric HCR cascade comprising more than two hairpin species (such as three, four, or five different HCR hairpin species) may be used. Configurations for HCR monomers and HCR reactions have been described in detail, for example, in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401).

An exemplary design of DNA hairpins as HCR monomers is shown in FIG. 4. The first HCR monomer species comprises a self-complementary hairpin with an interior loop and an input domain comprising a toehold at the 5′ end. The second HCR monomer species comprises a self-complementary hairpin with an interior loop and an input domain comprising a toehold at the 3′ end. The input domain of the first HCR monomer species is capable of hybridizing to the initiator sequence, and the output domain of the second HCR monomer species is capable of hybridizing to the terminator sequence. When the first and second monomer species are added to a biological sample comprising the initiator, the initiator hybridizes to the input domain of a monomer of the first HCR monomer species, thereby exposing its output domain, which hybridizes to the input domain of a monomer of the second species, thereby exposing the output domain of the monomer of the second species, which hybridizes to the input domain of another monomer of the first HCR species, and so on. In this way, alternating hybridization events between monomers of the first and second HCR monomer species propagate the HCR reaction to form a growing polymeric HCR product. The reaction is terminated when the output domain of an HCR monomer of the polymeric HCR product (shown as a monomer of the second HCR monomer species in FIG. 4), hybridizes to the terminator sequence. Hybridization of the output domain of an HCR monomer of the polymeric HCR product to the terminator sequence prevents hybridization of the output domain to another HCR monomer, thereby terminating the HCR reaction.

A number of criteria can be used to design the HPR monomers to achieve the desired properties. These include, for example and without limitation, sequence symmetry minimization, the probability of adopting the target secondary structure at equilibrium, the average number of incorrect nucleotides at equilibrium relative to the target structure, and hybridization kinetics.

### B. Initiator Sequence

The initiator sequence, as described herein, is a region or sequence of nucleic acid that is able to initiate an HPR reaction. In some aspects, the initiator sequence is comprised by the target nucleic acid molecule or by an adapter probe hybridized directly or indirectly to the target nucleic acid molecule. Thus, in some aspects, the presence and/or location of the initiator sequence is indicative of the presence and/or location of one or more of the target nucleic acid molecule.

In some embodiments, the initiator sequence comprises a region that is capable of hybridizing to (e.g., is complementary to) the input domain of an HPR monomer. In some embodiments, the initiator sequence is capable of hybridizing to the input domain of one or more of the HPR monomer species. In some embodiments, the HPR monomers polymerize in the presence of the initiator sequence. In some embodiments, the HPR monomers polymerize in a biological sample comprising the initiator sequence. In some embodiments, the presence of the polymeric HPR product is indicative of the presence of the initiator sequence, and thereby the target nucleic acid molecule. In some embodiments, the location of the polymeric HPR product is indicative of the location of the initiator sequence, and thereby the target nucleic acid molecule. In some embodiments, the number of polymeric HPR products is indicative of the number of initiator sequences, and thereby target nucleic acid molecules.

In some embodiments, the target nucleic acid comprises the initiator sequence. In other embodiments, the initiator sequence is comprised by an adapter probe further comprising a hybridization region that hybridizes directly or indirectly to the target nucleic acid molecule. In some embodiments, the adapter probe comprising the initiator sequence directly hybridizes to the target nucleic acid molecule. In some embodiments, the adapter probe comprising the initiator sequence indirectly hybridizes to the target nucleic acid molecule (e.g., via a branch probe or branch probe complex that hybridizes to the target nucleic acid molecule). In some embodiments, the initiator sequence comprised by an adapter probe can base-pair with an HPR monomer, e.g., of the first HPR monomer species. In some aspects, the initiator sequence is able to hybridize to an HPR monomer, triggering polymerization, when the adapter probe comprising the initiator sequence is hybridized directly or indirectly to the target nucleic acid molecule.

In some embodiments, the adapter probe comprising the initiator sequence comprises one or more of the following: DNA, RNA, 2′Ome-RNA, LNA, synthetic nucleic acid analog, amino acid, synthetic amino acid analog, and PNA.

In some embodiments, the adapter probe comprising the initiator sequence is provided in one part. In some embodiments, the adapter probe comprising the initiator sequence is provided in more than one part. In some embodiments, the adapter probe comprising the initiator sequence comprises a split adapter probe, such as the split adapter probe shown in FIG. 2F. FIG. 2F shows an exemplary configuration in which the terminator sequence is comprised by a first adapter probe hybridized to the target nucleic acid, and the initiator sequence is provided in a split adapter probe. The split adapter probe comprises a first and second adapter oligonucleotide. The first adapter oligonucleotide comprises a first portion of the initiator sequence and the second adapter oligonucleotide comprises a second portion of the initiator sequence. The first and second adapter oligonucleotides hybridize to adjacent sequences on the target nucleic acid molecule, or to sequences separated by fewer than about 100 intervening nucleotides on the target nucleic acid molecule. In some embodiments, the first and second adapter oligonucleotides hybridize to sequences separated by fewer than about 1000 intervening nucleotides, fewer than about 500 intervening nucleotides, fewer than about 400 intervening nucleotides, fewer than about 200 intervening nucleotides, fewer than about 100 intervening nucleotides, fewer than about 50 intervening nucleotides, fewer than about 40 intervening nucleotides, fewer than about 30 intervening nucleotides, or fewer than about 20 intervening nucleotides, on the target nucleic acid molecule. Hybridization of the first and second adapter oligonucleotides to the target nucleic acid molecule brings the first and second portion of the initiator sequence into proximity, thereby constituting an initiator sequence that is able to hybridize to an HPR monomer, thereby initiating an HPR reaction.

### C. Terminator Sequence

The terminator sequence, as described herein, is a region or sequence of nucleic acid that is capable of terminating an HPR reaction. In some embodiments, the binding of a complementary sequence to the terminator sequence inhibits an HPR monomer and/or portion thereof from hybridizing to another HPR monomer, thus halting further progress and polymerization in the hybridization complex. In some aspects, the terminator sequence is comprised by the target nucleic acid molecule or by an adapter probe hybridized directly or indirectly to the target nucleic acid molecule. In some aspects, in the absence of a terminator sequence, an HPR reaction may proceed for as long as HPR monomers are available. In some aspects, hybridization of an HPR monomer of a polymeric HPR product to the terminator sequence terminates the HPR reaction. Thus, in some aspects, the presence of the terminator sequence can reduce the size of (i.e. number of HPR monomers in) a polymeric HPR product, or the average size or the dispersity of polymeric HPR products in a biological sample in which multiple HPR reactions were initiated. In some aspects, the presence of the terminator sequence can control and terminate the amplification reaction (e.g., HPR reaction) such that further amplification (e.g., incorporation of additional HPR monomers) is halted.

In some embodiments, the terminator sequence comprises a nucleic acid sequence that hybridizes to an HPR monomer. In some embodiments, the terminator sequence comprises a nucleic acid sequence that is complementary to an HPR monomer. In some embodiments, an HPR monomer of the polymeric HPR product hybridizes to the terminator sequence. In some embodiments, a portion, such as the output domain, of the HPR monomer of the polymeric HPR product hybridizes to the terminator sequence. In some embodiments, the hybridization of the portion of the HPR monomer of the polymeric HPR product to the terminator sequence inhibits the HPR monomer and/or portion thereof from hybridizing to another HPR monomer. In some embodiments, the portion of the HPR monomer of the polymeric HPR product hybridized to the terminator sequence has a higher melting temperature than the portion of the HPR monomer of the polymeric HPR product hybridized to another HPR monomer. In some embodiments, the portion of the HPR monomer of the polymeric HPR product has greater complementarity to the terminator sequence than to another HPR monomer. In some embodiments, the hybridization of the HPR monomer of the polymeric HPR product to the terminator sequence terminates the HPR.

In some embodiments, the terminator sequence comprises deoxyribonucleic acid (DNA), ribonucleic acid (RNA) and/or locked nucleic acid (LNA).

In some embodiments, the target nucleic acid comprises the terminator sequence. In some embodiments, the terminator sequence is comprised by an adapter probe further comprising a hybridization region that hybridizes directly or indirectly to the target nucleic acid molecule. In some embodiments, the adapter probe comprising the terminator sequence directly hybridizes to the target nucleic acid molecule. In some embodiments, the adapter probe comprising the terminator sequence indirectly hybridizes to the target nucleic acid molecule (e.g., via a branch probe that hybridizes to the target nucleic acid molecule or a branch probe complex that hybridizes to the target nucleic acid molecule). In some embodiments, the terminator sequence can base-pair with an HPR monomer of the polymeric HPR product (e.g., via the output domain of the HPR monomer). In some aspects, the terminator sequence is able to hybridize to an HPR monomer of the polymeric HPR product, when the adapter probe comprising the terminator sequence is hybridized directly or indirectly to the target nucleic acid molecule.

In some embodiments, the adapter probe comprising the terminator sequence comprises one or more of the following: DNA, RNA, 2′Ome-RNA, LNA, synthetic nucleic acid analog, amino acid, synthetic amino acid analog, and PNA.

In some embodiments, the adapter probe comprising the terminator sequence comprises a hybridization region that hybridizes to the target nucleic acid molecule or to a branch probe or branch probe complex that hybridizes to the target nucleic acid molecule. In some embodiments, the target nucleic acid comprises the terminator sequence (e.g., as shown in FIGS. 2A and 2B). In some embodiments, the target nucleic acid is an endogenous molecule such as a DNA or RNA comprising a terminator sequence. In some embodiments, an endogenous sequence in an RNA is an initiator sequence. In some embodiments, an endogenous sequence in an RNA is a terminator sequence. In some embodiments, the target nucleic acid is a probe that directly or indirectly binds to an endogenous molecule such as a DNA or RNA, and the probe comprises an initiator sequence and/or a terminator sequence. In some embodiments, the target nucleic acid comprises both the initiator sequence and the terminator sequence. In some embodiments, the target nucleic acid is an endogenous nucleic acid analyte (e.g., DNA or RNA), a product of an endogenous nucleic acid analyte, a probe that directly or indirectly binds to an endogenous nucleic acid analyte, or a product of a probe that directly or indirectly binds to an endogenous nucleic acid analyte. In some embodiments, the terminator sequence is comprised by an adapter probe comprising the terminator sequence. In some embodiments, the adapter probe comprising the terminator sequence comprises a nucleic acid sequence that is complementary to the target nucleic acid. In some embodiments, the terminator sequence is comprised by one or more nucleic acid probes (i.e., adapter probes) that hybridize directly or indirectly to the target nucleic acid (e.g., as illustrated in FIGS. 2C-2F and FIGS. 3A-3E). In some embodiments, the initiator sequence and the terminator sequence are comprised by separate adapter probes (e.g., as shown in FIG. 2D). In some embodiments, a first adaptor probe comprises the initiator sequence and a second adapter probe comprises the terminator sequence. In some embodiments, both the initiator sequence and terminator sequence are comprised by the same adapter probe (e.g., as shown in FIG. 2E).

In some embodiments, the terminator sequence is comprised by a terminator oligonucleotide that does not comprise a separate sequence for hybridization directly or indirectly to the target nucleic acid molecule.

A number of criteria can be used to design the HPR monomers to achieve the desired properties. These include, for example and without limitation, the average number of mismatched nucleotides between terminator-HPR monomer hybridization relative to HPR monomer-monomer hybridization and/or initiator-HPR monomer hybridization, and hybridization thermodynamics and kinetics. For example, in some embodiments, the HPR monomer hybridized to the terminator sequence has a higher melting temperature than the HPR monomer hybridized to another HPR monomer. In some embodiments, the terminator sequence comprises deoxyribonucleic acid (DNA), ribonucleic acid (RNA) and/or locked nucleic acid (LNA).

The terminator sequence may be provided at any desired point during the HPR reaction, such as prior to the addition of any other probes, simultaneously with some of the probes, or after addition of all other probes. In some embodiments, the method provided herein comprises contacting the biological sample with the plurality of HPR monomers and one or more adapter probes comprising the terminator sequence, simultaneously or sequentially in any order. The adapter probe comprising the terminator sequence may also be complexed with one or more additional probes, for example as described below.

### D. HPR Configurations

In some embodiments, the initiator sequence and terminator sequence are each independently comprised by the target nucleic acid molecule or by an adapter probe hybridized directly or indirectly to the target nucleic acid molecule. In some embodiments, the proximity of the terminator sequence to the initiator sequence allows the terminator sequence to terminate the HPR reaction initiated by the initiator sequence. In some embodiments, the number, proximity, and/or arrangement of one or more terminator sequences relative to the initiator sequence influences the resulting size and/or dispersity of polymeric HPR products in the biological sample. In some embodiments, increasing the number of terminator sequences, and/or the proximity of terminator sequences to the initiator sequence reduces the resulting size and/or dispersity of polymeric HPR products. Various arrangements of initiator sequences and terminator sequences for facilitating an HPR reaction that is terminated by a terminator sequence are described below, and with reference to the drawings.

In some aspects, in the absence of a terminator sequence, an HPR reaction may proceed for as long as HPR monomers are available. In contrast, in some aspects, hybridization of an HPR monomer of a polymeric HPR product to the terminator sequence can inhibit further polymerization and terminate the HPR reaction. This is exemplified in FIGS. 1A-D.

FIGS. 1A-1D show schematics illustrating exemplary HPR reactions with or without a terminator sequence.

FIG. 1A shows an exemplary HPR comprising a plurality of monomers of two HPR monomer species, which is initiated by an initiator sequence. The initiator sequence hybridizes to the input domain of a monomer of the first HPR monomer species. The output domain of the monomer of the first HPR monomer species hybridizes to the input domain of a monomer of the second HPR monomer species. The output domain of the monomer of the second HPR monomer species in turn hybridizes to the input domain of another monomer of the first HPR monomer species, which itself in turn hybridizes via its output domain to the input domain of another monomer of the second HPR monomer species. Repeated hybridization events between the two monomer species can sustain the HPR for as long as monomers are available.

FIG. 1B shows an exemplary HPR comprising a plurality of monomers of two HPR monomer species, which is initiated by an initiator sequence and terminated by a terminator sequence. The reaction is initiated and sustained as in FIG. 1A, until a portion of an HPR monomer of the polymeric HPR product hybridizes to the terminator sequence, for example via its output domain. Hybridization of the portion of the HPR monomer of the polymeric HPR product to the terminator sequence inhibits the output domain from hybridizing to another HPR monomer, thereby inhibiting further polymerization. The portion of the HPR monomer of the polymeric HPR product hybridized to the terminator sequence may have a higher melting temperature than the portion of the HPR monomer of the polymeric HPR product hybridized to another HPR monomer. The portion of the HPR monomer of the polymeric HPR product may also have greater complementarity to the terminator sequence than to another HPR monomer.

FIG. 1C shows an exemplary HPR comprising a plurality of monomers of two HPR monomer species, which is initiated by an initiator sequence and terminated by a terminator sequence. The initiator sequence and terminator sequence are each comprised by a single target nucleic acid molecule.

FIG. 1D shows an exemplary HPR comprising a plurality of monomers of two HPR monomer species, which is initiated by an initiator sequence and terminated by a terminator sequence. The initiator sequence is comprised by a first adapter probe that hybridizes to the target nucleic acid molecule, and the terminator sequence is comprised by a second adapter probe that hybridizes to the target nucleic acid molecule.

In some embodiments, the initiator sequence and the terminator sequence can each independently be comprised by an adapter probe that hybridizes directly or indirectly to the target nucleic acid molecule. In some embodiments, the initiator sequence and terminator sequence are comprised by the same adapter probe. In some embodiments, the initiator sequence and terminator sequence are comprised by different adapter probes. Various exemplary configurations of the initiator sequence and terminator sequence are shown in FIGS. 2A-2F.

FIGS. 2A-2F show schematics illustrating exemplary configurations of a target nucleic acid, initiator sequence, and terminator sequence for an HPR reaction. The HPR monomers and polymeric HPR product are not shown.

FIG. 2A shows an exemplary configuration in which the initiator sequence and terminator sequence are each comprised by the target nucleic acid. FIG. 2B shows an exemplary configuration in which the initiator sequence is comprised by an adapter probe hybridized to the target nucleic acid, and the terminator sequence is comprised by the target nucleic acid. FIG. 2C shows an exemplary configuration in which the initiator sequence is comprised by the target nucleic acid, and the terminator sequence is comprised by an adapter probe hybridized to the target nucleic acid. FIG. 2D shows an exemplary configuration in which the initiator sequence is comprised by a first adapter probe hybridized to the target nucleic acid, and the terminator sequence is comprised by a second adapter probe hybridized to the target nucleic acid. FIG. 2E shows an exemplary configuration in which the initiator sequence and the terminator sequence are each comprised by the same adapter probe, which is hybridized to the target nucleic acid. FIG. 2F shows an exemplary configuration in which the terminator sequence is comprised by a first adapter probe hybridized to the target nucleic acid, and the initiator sequence is provided in a split adapter probe. The split adapter probe comprises a first and second adapter oligonucleotide. The first adapter oligonucleotide comprises a first portion of the initiator sequence and the second adapter oligonucleotide comprises a second portion of the initiator sequence. In some embodiments, the first and second adapter oligonucleotides hybridize to adjacent sequences on the target nucleic acid molecule, or to sequences separated by fewer than about 100 intervening nucleotides on the target nucleic acid molecule. Hybridization of the first and second adapter oligonucleotides to the target nucleic acid molecule brings the first and second portion of the initiator sequence into proximity, thereby constituting an initiator sequence that is able to hybridize to an HPR monomer, thereby initiating an HPR reaction.

In some embodiments, multiple copies of the terminator sequence are provided. In some embodiments, an adapter probe comprising the terminator sequence comprises multiple copies of the terminator sequence. In some embodiments, a plurality of adapter probes comprising the terminator sequence hybridize to the target nucleic acid or to a branch probe or branch probe complex that hybridizes to the target nucleic acid molecule. Any desired number of adapter probes comprising the terminator sequence may hybridize to the branch probe or branch probe complex. In some embodiments, at least 2, at least 5, at least 10, at least 15, or at least 20 adapter probes comprising the terminator sequence hybridize to a branch probe or branch probe complex that is hybridized to the target nucleic acid. In some embodiments, a plurality of branch probes or branch probe complexes are each hybridized to (i) a plurality of adapter probes comprising the terminator sequence, and (ii) the target nucleic acid molecule. In some embodiments, the branch probe complex is a branched hybridization complex.

Various exemplary configurations of the initiator sequence and multiple terminator sequences are shown in FIGS. 3A-3D.

FIGS. 3A-3D show schematics illustrating exemplary configurations of a target nucleic acid molecule, initiator sequence, and terminator sequence for an HPR, in which multiple copies of the terminator sequence are provided. The HPR monomers and polymeric HPR product are not shown.

FIG. 3A shows an exemplary configuration in which a first adapter probe hybridized to the target nucleic acid comprises the initiator sequence, and a second adapter probe hybridized to the target nucleic acid comprises multiple copies of the terminator sequence.

FIG. 3B shows an exemplary configuration in which a first adapter probe hybridized to the target nucleic acid comprises the initiator sequence, and a plurality of adapter probes, each comprising the terminator sequence, are hybridized to a branch probe which is hybridized to the target nucleic acid.

FIG. 3C shows an exemplary configuration in which a first adapter probe hybridized to the target nucleic acid comprises the initiator sequence, and a plurality of adapter probes, each comprising the terminator sequence, are hybridized to a branch probe complex which is hybridized to the target nucleic acid. As shown, a branch probe complex described herein may comprise a branched hybridization structure.

FIG. 3D shows an exemplary configuration in which a plurality of adapter probes, each comprising the terminator sequence, are hybridized to a branch probe complex which is hybridized to the target nucleic acid. A separate adapter probe comprising the initiator sequence is also hybridized to the branch probe complex. In some embodiments, an adapter probe comprising the initiator sequence and an adapter probe comprising the terminator sequence hybridize to different branch probes and/or branch probe complexes. In some embodiments, an adapter probe comprising the initiator sequence and an adapter probe comprising the terminator sequence hybridize to the same branch probe and/or branch probe complex hybridized to the target nucleic acid.

In some embodiments, the initiator sequence and terminator sequence(s) are comprised by the same adapter probe, or comprised by adapter probes that hybridize to the same branch probe or branch probe complex. In such embodiments, the relative arrangement of initiator sequence and terminator sequence(s) can remain relatively constant when different target nucleic acids are targeted. In some aspects, this allows detection of different target nucleic acids using a similar HPR reaction.

FIG. 3E shows an exemplary configuration in which a first adapter probe hybridized to the target nucleic acid comprises the initiator sequence, and a plurality of branch probe complexes are hybridized to the target nucleic acid, each branch probe complex being hybridized to a plurality of adapter probes comprising the terminator sequence.

In some embodiments, the methods provided herein further comprise providing one or more additional probes. In some embodiments, provided are branch probes and/or branch probe complexes. In some embodiments, the branch probes and/or branch probe complexes are hybridized to the target nucleic acid. In some embodiments, a branch probe or branch probe complex is a probe or probe complex which hybridizes to the target nucleic acid and to an adapter probe comprising the initiator sequence and/or the terminator sequence. A branch probe may also be a probe within a branch probe complex, and may not itself hybridize directly to the adapter probe and/or the target nucleic acid. Thus, in some embodiments, a branch probe or branch probe complex facilitates indirect hybridization of an adapter probe comprising the initiator sequence and/or terminator sequence to the target nucleic acid, for example as described herein for FIGS. 3B-3E. Branch probes and branch probe complexes provide potential advantages such as increasing local effects and allowing for various probe designs.

### E. Additional Probe Considerations

In some embodiments, provided herein is a nucleic acid probe set comprising at least two or more probes (e.g., the first HPR monomer species, the second HPR monomer species, adapter probe(s) comprising the initiator sequence and/or terminator sequence, and/or branch probes) that can be joined to form a probe complex.

In some embodiments, more than one type of secondary nucleic acid probes may be contacted with a sample, e.g., simultaneously or sequentially in any suitable order, such as in sequential probe hybridization/unhybridization cycles. In some embodiments, more than one type of secondary probes may be contacted with a sample, e.g., simultaneously or sequentially in any suitable order, such as in sequential probe hybridization/unhybridization cycles. In some embodiments, more than one type of higher order nucleic acid probes may be contacted with a sample, e.g., simultaneously or sequentially in any suitable order, such as in sequential probe hybridization/unhybridization cycles. In some embodiments, more than one type of detectably labeled nucleic acid probes may be contacted with a sample, e.g., simultaneously or sequentially in any suitable order, such as in sequential probe hybridization/unhybridization cycles. In some embodiments, the detectably labeled probes may comprise probes that bind to one or more primary probes, one or more secondary probes, one or more higher order probes, between a primary/second/higher order probes, and/or one or more detectably or non-detectably labeled probes (e.g., as in the case of a hybridization-based polymerization reaction (HPR) such as a hybridization chain reaction (HCR)). In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., primary, secondary, higher order probes, and/or detectably labeled probes) can be contacted with a sample, e.g., simultaneously or sequentially in any suitable order.

Between any of the probe contacting steps disclosed herein, the method may comprise one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The target-binding sequence (sometimes also referred to as the hybridization region, targeting region/sequence or the recognition region/sequence) of a probe may be positioned anywhere within the probe. For instance, the hybridization region of an adapter probe that binds to a target nucleic acid can be 5′ or 3′ to any other sequence in the probe, such as an initiator sequence and/or terminator sequence. In some embodiments, the hybridization region may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some embodiments, a nucleic acid probe (e.g. an HPR monomer or product thereof, or a probe hybridized directly or indirectly thereto) disclosed herein may contain a detectable label such as a fluorophore. In some embodiments, one or more probes of a plurality of nucleic acid probes used in an assay may each comprise a detectable label selected from a limited pool of distinct detectable labels (e.g., red, green, yellow, and blue fluorophores). In some embodiments, a nucleic acid probe or probe set disclosed herein lacks a detectable label.

In some embodiments, a nucleic acid probe herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order.

In some embodiments, nucleic acid probes disclosed herein (e.g., branch probes or branch probe complexes and adapter probes comprising initiator sequences and/or terminator sequences) can be pre-assembled from multiple components, e.g., prior to contacting the nucleic acid probes with the biological sample. In some embodiments, nucleic acid probes disclosed herein can be assembled during and/or after contacting a target nucleic acid or a sample with multiple components. In some embodiments, nucleic acid probes disclosed herein are assembled in situ in a sample. In some embodiments, the nucleic acid probes can be contacted with a target nucleic acid or a sample in any suitable order and any suitable combination. In some embodiments, the nucleic acid probes can be assembled in situ in a stepwise manner, each step with the addition of one or more components, or in a dynamic process where all components are assembled together. One or more removing steps, e.g., by washing the sample such as under stringent conditions, may be performed at any point during the assembling process, for example to remove undesired components (e.g., unbound adapter probes).

In some embodiments, the branch probe complex comprises multiple amplifier probes bound to sites in a pre-amplifier probe. In some embodiments, the pre-amplifier probe comprises a sequence that is complementary to a sequence in the target nucleic acid. In some embodiments, a pre-amplifier probe binds to one or more probes that hybridize to the target nucleic acid. In some embodiments, the pre-amplifier probe comprises a sequence that is complementary to an adapter probe comprising a terminator sequence. In some embodiments, the pre-amplifier probe is capable of binding simultaneously to one or more adapter probe molecules and to a target nucleic acid molecule. In some embodiments, an amplifier probe comprises a sequence that is complementary to a sequence in a pre-amplifier probe. In some embodiments, an amplifier probe binds to one or more probes that directly or indirectly bind to a pre-amplifier probe. In some embodiments, an amplifier probe comprises a sequence that is complementary to an adapter probe comprising a terminator sequence. In some embodiments, an amplifier probe is capable of binding simultaneously to one or more adapter probe molecules and to a pre-amplifier probe.

### IV. HYBRIDIZATION-BASED POLYMERIZATION REACTIONS (HPRS)

In some embodiments, the HPR is an HCR. In some embodiments of HCR, two different HCR monomer species provided as DNA hairpins (e.g., as described above for FIG. 4) undergo a chain reaction of hybridization events to form a polymeric HCR product when contacted by an initiator sequence. In the absence of the initiator sequence the hairpin monomers are stable, or kinetically trapped (“metastable”), and remain as monomers. Hybridization of the initiator sequence to the input domain a first HCR monomer initiates the HCR reaction, opening the first HCR monomer and allowing its output domain to hybridize to the input domain of a second HCR monomer, and so on, leading to the formation of a polymeric HCR product comprising a chain of alternating units of the first and second monomer species in a nicked double helix.

In some embodiments, HCR reactions can be carried out with more than two HCR monomer species. For example, a system involving a first, second, and third HCR monomer species could be used. In some embodiments of such a system, the first HCR monomer species comprises an output domain which hybridizes to the input domain of a second HCR monomer species; the second HCR monomer species comprises an output domain which hybridizes to the input domain of a third HCR monomer species; and the third HCR monomer species comprises an output domain which hybridizes to the input domain of the first HCR monomer species. The HCR reaction can be initiated by an initiator sequence that hybridizes to the input domain of one of the HCR monomer species, and the HCR reaction can proceed generally as described above, except that the resulting product is a polymeric HCR product having a repeating unit of first, second, and third HCR monomer species, consecutively. Corresponding systems with larger numbers of HCR monomer species can also be used.

In some embodiments, the HPR reaction may comprise a linear oligo hybridization chain reaction (LO-HCR). In some embodiments, one or more of the HPR monomer species does not comprise a hairpin structure. In some embodiments, one or more of the HPR monomer species does not comprise a metastable secondary structure. In some embodiments, the LO-HCR polymer does not comprise a branched structure. Exemplary methods and compositions for LO-HCR are described in US 2021/0198723, which is incorporated herein by reference in its entirety.

In some embodiments, the method may comprise contacting the biological sample with any of the components described herein simultaneously or sequentially, in any order. In some embodiments, the biological sample is contacted with the first HPR monomer species, the second HPR monomer species, and one or more adapter probes comprising the initiator sequence and/or terminator sequence simultaneously or sequentially, in any order. One or more additional components, such as a branch probe or branch probe complex, or a terminator oligonucleotide, may also be added simultaneously or sequentially in any order. For example, an adapter probe comprising the initiator sequence and a separate adapter probe comprising the terminator sequence is introduced to the sample, followed by a wash to remove unbound adapter probes, and then the HPR monomers (e.g. the first and the second HPR monomer species) are introduced to the sample. In some embodiments, the branch probes and/or branch probe complexes are hybridized to the target nucleic acid molecule prior to contacting the sample with the plurality of HPR monomers and/or adapter probes. In some embodiments, one or more adapter probe comprising the terminator sequence is hybridized to a branch probe or branch probe complex, and the branch probe or branch probe complex is hybridized to the target nucleic acid (and optionally an adapter probe comprising the initiator sequence) prior to contacting the sample with the plurality of HPR monomers. Various other orders and combinations are contemplated and may be used.

In some embodiments, the method comprises contacting the biological sample with a terminator oligonucleotide which comprise the terminator sequence and which does not comprise a separate sequence for hybridization directly or indirectly to the target nucleic acid molecule. In some embodiments, the addition of the terminator oligonucleotide terminates HPR reactions that have yet to be terminated by a terminator sequence that is comprised by an adapter probe or by the target nucleic acid.

In some embodiments, the method further requires incubation upon addition of one or more probes. In some embodiments, incubating results in binding (e.g., hybridization) of a probe to its target (e.g., hybridization of an adapter probe to the target nucleic acid) In some embodiments, incubating occurs at room temperature. In some embodiments, incubating occurs at 4° C. In some embodiments, incubating occurs at 37° C. In some embodiments, incubating occurs at 45° C. In some embodiments, incubating occurs at 50° C. In some embodiments, incubating occurs at 55° C. In some embodiments, incubating occurs at 60° C. In some embodiments, incubating comprises an incubation period of at least 1 minute, for example, 5 minutes, 15 minutes, 30 minutes or 1 hour. In some embodiments, the incubation period exceeds 1 hour, for example, 2 hours, 4 hours, 12 hours, 16 hours, or 24 hours. In some embodiments, incubating occurs in hybridization buffer containing 0% formamide. In some embodiments, incubating occurs in hybridization buffer comprising formamide. In some embodiments, the percent concentration of formamide is between 1% and 80%, for example, between 10% and 70%, or between 30% and 60%. In some embodiments, the hybridization buffer comprises citric acid. In some embodiments, the molar concentration of citric acid is between 1 nM and 30 nM, for example, between 5 nM and 15 nM or between 8 nM and 12 nM. In some embodiments, the hybridization buffer comprises Tween. In some embodiments, the percent concentration of Tween is between 0% and 1.0%, for example, between 0.05% and 0.5%. In some embodiments, the hybridization buffer comprises heparin. In some embodiments, the concentration of heparin is between 20 μg/mL and 80 μg/mL, for example, between 30 μg/mL and 70 μg/mL, for example, between 40 μg/mL and 60 μg/mL. In some embodiments, the hybridization buffer comprises Denhardt's solution. In some embodiments, the hybridization buffer comprises dextran sulfate. In some embodiments, the percent concentration of dextran sulfate is between 1% and 60%, for example, between 40% and 60%.

In some aspects, the provided methods involve analyzing, e.g., detecting or determining, one or more sequences present in the probes (e.g., hybridized first and second HPR monomer species) and/or in a product or derivative thereof. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence present in the sample. In some embodiments, the analysis includes quantification of puncta. In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some embodiments, the analysis further includes displaying the information from the analysis or detection step. In some embodiments, software may be used to automate the processing, analysis, and/or display of data.

In some embodiments, a method disclosed herein may also comprise one or more signal amplification components. In some embodiments, the one or more signal amplification components are used to detect signals associated with the generated amplification product (e.g., polymeric HPR product). In some embodiments, the present disclosure relates to the detection of nucleic acids sequences in situ using probe hybridization and generation of amplified signals associated with the probes, wherein background signal is reduced and sensitivity is increased.

### V. DETECTION AND ANALYSIS

In some embodiments, the methods comprise detection and analysis of an HPR product. In some embodiments, the methods comprise detection and analysis of one or more polymeric HPR products in a biological sample. In some embodiments, the methods comprise determining a sequence of all or a portion of an amplification product (e.g., the polymeric HPR product). In some embodiments, detection of the HPR product is indicative of the presence of the target nucleic acid.

In some embodiments, the detection, analysis and/or sequence determination comprises detecting a polymer generated by a hybridization chain reaction (HCR) reaction, see e.g., US 2017/0009278, which is incorporated herein by reference. In some embodiments, the detection or determination comprises hybridizing to the product a detection oligonucleotide labeled with a detectable label, e.g., a fluorophore, an isotope, a mass tag, or a combination thereof. In some embodiments, the detection or determination comprises imaging the product. In some embodiments, the target nucleic acid is an mRNA in a tissue sample, and the detection or determination is performed when the target nucleic acid and/or the product is in situ in the tissue sample.

In some embodiments, one or more of the HPR monomers is labeled with a detectable label, e.g., a fluorophore, isotope, mass tag, or combination thereof. In some embodiments, the detectable label is a fluorescent label. In some embodiments, detecting the polymeric HPR product comprises detecting a signal generated from the detectable label. In some embodiments, the detectable label can be measured and quantitated. In some embodiments, the intensity of the signal generated is proportional to the size of the HPR product (i.e. number of HPR monomers in the polymeric HPR product). In some embodiments, the number of signals detected in the biological sample is indicative of the number of target nucleic acids present in the biological sample.

In some embodiments, one or more of the HPR monomers can include a reporter molecule, such that polymerization of the HPR monomers (first and second, and optionally more), will result in a detectable signal. In some embodiments, the reporter molecule can be covalently associated with the hairpin monomer(s). In some embodiments, the reporter molecule can be subsequently bound to the HPR polymer after polymerization (e.g., in a subsequent hybridization event). In some embodiments, the first and/or the second HPR monomer comprises a label-binding site that is configured to hybridize to a sequence complementary to the label-binding site. In some embodiments, the sequence complementary to the label-binding site further comprises a reporter molecule.

In some embodiments, the identification of polymeric HPR products comprising the first and second monomer species is indicative of the presence of the analyte in the sample. In some embodiments, polymeric HPR products may be identified, for example, by gel electrophoresis.

In some embodiments, two HPR monomer species (e.g., the first and second HPR monomer species) form a donor-acceptor pair for fluorescence resonance energy transfer (FRET) pair when combined as a polymer. In some embodiments, the FRET pair is formed as a consequence of a change in quenching or FRET that occurs when the HPR monomers open and polymerize. Thus, in some embodiments, the HPR monomers are configured to allow for FRET (e.g., meeting proximity requirements and reporter molecule pairing requirements for changes in FRET to be monitored), and FRET imaging can be used to detect and analyze polymeric HCR products. The use of FRET to detect HPR polymerization is described, for example, in Huang et al., Chem. Sci. 7(6):3829-3835, 2016.

In some aspects, the terms “label” and “detectable label” comprise a directly or indirectly detectable moiety that is associated with (e.g., conjugated to) a molecule to be detected, e.g., a detectable probe (such as an HPR monomer), comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon or polymeric HPR product) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345, which is incorporated herein by reference in its entirety.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detection, analysis and/or sequence determination can be carried out at room temperature for best preservation of tissue morphology with low background noise and error reduction. In some embodiments, the detection, analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of a detectable label (e.g., a detectable label comprised by a polymeric HPR product). In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of a detectable label (e.g., a detectable label comprised by a polymeric HPR product). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, a product herein includes a molecule or a complex generated in a series of reactions, e.g., hybridization, ligation, extension, replication, transcription/reverse transcription, and/or amplification in any suitable combination.

In some embodiments, the method further comprises identifying multiple different target nucleic acids present at locations (e.g., different locations) in the biological sample. In some embodiments, each different target nucleic acid is associated with a different detectable label. In some embodiments, each different target nucleic acid comprises or is directly or indirectly hybridized to a different initiator sequence, wherein each initiator sequence is capable of initiating an HPR reaction comprising different HPR monomer species and/or different detectable labels. In some embodiments, different HPR monomer species and/or detectable labels facilitate analysis of different target nucleic acids, which may be present in the same biological sample. In some embodiments, different target nucleic acids are associated with the same detectable label, and can be detected and analyzed together (e.g., simultaneously) or separately (e.g., sequentially).

### VI. COMPOSITIONS AND KITS

Also provided herein are compositions and kits, for example comprising one or more polynucleotides, e.g., any described in Section III, and reagents for performing the methods provided herein, for example reagents required for one or more steps comprising hybridization, ligation, amplification, detection, sequencing, and/or sample preparation as described herein. In some embodiments, the composition or kit further comprises a target nucleic acid, e.g., any described in Section II. In some embodiments, any or all of the polynucleotides are DNA molecules. In some embodiments, the target nucleic acid is an RNA molecule, such as a messenger RNA molecule. In some embodiments, the composition or kit further comprises one or more ligases. In some embodiments, the composition or kit further comprises a polymerase. In some embodiments, the composition or kit further comprises one or more detection reagents such as those disclosed in Section V.

In some embodiments, disclosed herein is a composition or kit for analyzing a biological sample according to any of the methods described herein. In some embodiments, the composition or kit comprises a plurality of HPR monomers comprising a first HPR monomer species and a second HPR monomer species, wherein: the plurality of HPR monomers are configured to (a) polymerize to generate a polymeric HPR product when a monomer of the first HPR monomer species hybridizes to an initiator sequence, and (b) terminate polymerization when a monomer of the HPR product hybridizes to a terminator sequence; the first HPR monomer species and second HPR monomer species are each capable of hybridizing to another HPR monomer species; and the initiator sequence and terminator sequence are each independently comprised by (i) a target nucleic acid molecule, or (ii) an adapter probe hybridized directly or indirectly to the target nucleic acid molecule. In some embodiments, the composition or kit further comprises an adapter probe comprising the initiator sequence, and/or an adapter probe comprising the terminator sequence. In some embodiments, the composition or kit further comprises an adapter probe comprising the initiator sequence and the terminator sequence. In some embodiments, the composition or kit further comprises one or more of a branch probe or a branch probe complex, wherein each branch probe or branch probe complex is capable of hybridizing to (a) the target nucleic acid molecule, and (b) an adapter probe comprising the initiator and/or terminator sequence.

In some embodiments, disclosed herein is a kit comprising a plurality of HPR monomers of a first HPR monomer species; a plurality of HPR monomers of a second HPR monomer species, wherein the first HPR monomer species and the second HPR monomer species are each capable of binding to another HPR monomer species to generate an amplification product (e.g., a polymeric HPR product); an adapter probe comprising an initiator sequence, and/or an adapter probe comprising a terminator sequence, wherein a monomer of the HPR product comprises a sequence complementary to the terminator sequence. In some embodiments, disclosed herein is a kit comprising a plurality of HPR monomers of a first HPR monomer species; a plurality of HPR monomers of a second HPR monomer species, wherein the first HPR monomer species and the second HPR monomer species are each capable of binding to another HPR monomer species to generate an amplification product (e.g., a polymeric HPR product); and an adapter probe comprising a initiator sequence and a terminator sequence, wherein a monomer of the HPR product comprises a sequence complementary to the terminator sequence.

In some embodiments, the composition or kit further comprises the biological sample. In some embodiments, the biological sample is fixed. In some embodiments, the biological sample is unfixed. In some embodiments, the biological sample is permeabilized. In some embodiments, the biological sample is embedded in a matrix. In some embodiments, the matrix comprises a hydrogel. In some embodiments, the biological sample is cleared. In some embodiments, the clearing comprises contacting the biological sample with a proteinase. In some embodiments, the biological sample is crosslinked. In some embodiments, the biological sample is a tissue slice. In some embodiments, the tissue slice is between about 1 μm and about 50 μm, about 2 μm and about 50 μm, about 3 μm and about 50 μm, about 4 μm and about 50 μm, about 5 μm and about 50 μm, about 5 μm and about 45 μm, about 5 μm and about 40 μm, about 5 μm and about 35 μm, about 5 μm and about 30 μm, about 5 μm and about 25 μm, about 6 μm and about 20 μm, about 7 μm and about 15 μm, or about 8 μm and about 10 μm in thickness. In some embodiments, the tissue slice is between about 5 μm and about 35 μm in thickness.

The various components of the kit may be present in separate

containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect regions of interest in target nucleic acids.

In some embodiments, the region of interest comprises a single-nucleotide polymorphism (SNP). In some embodiments, the region of interest comprises is a single-nucleotide variant (SNV). In some embodiments, the region of interest comprises a single-nucleotide substitution. In some embodiments, the region of interest comprises a point mutation. In some embodiments, the region of interest comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VII. TERMINOLOGY

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample and/or a probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences.

Barcodes can spatially-resolve molecular components found in biological samples, for example, at single-cell resolution. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of the sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

Two nucleic acid sequences can become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension and Polymerization

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

As used herein, “polymerization” refers to the association of two or more monomers to form a polymer. The “polymer” may comprise covalent bonds, non-covalent bonds or both. For example, in some embodiments two species of monomers are able to hybridize in an alternating pattern to form a polymer comprising a nicked double helix. The polymers are also referred to herein as “HCR products.”

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 90N) DNA ligase (90NTM DNA ligase, available from New England Biolabs, Ipswich, MA), and Ampligase™ (available from Epicentre Biotechnologies, Madison, WI). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a probe associated with a feature. For example, detectably labelled features can include a fluorescent, a colorimetric, or a chemiluminescent label (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DIA (4-Di-16-ASP), DiD (DilC18(5)), DIDS, Dil (DilC18(3)), DiO (DiOC18(3)), DIR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), Lyso Tracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families can provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### VIII. EXEMPLARY EMBODIMENTS

Among the provided embodiments are:

1. A method for analyzing a biological sample, comprising:
(a) contacting the biological sample with a plurality of hybridization-based polymerization reaction (HPR) monomers comprising a first and a second HPR monomer species, wherein hybridization of a monomer of the first HPR monomer species to an initiator sequence initiates an HPR reaction to generate a polymeric HPR product, wherein:
   the first HPR monomer species and the second HPR monomer species are each capable of hybridizing to another HPR monomer species;
   at least one of the HPR monomer species is capable of hybridizing to a terminator sequence; and
   the initiator sequence and terminator sequence are each independently comprised by (i) a target nucleic acid molecule, or (ii) an adapter probe hybridized directly or indirectly to the target nucleic acid molecule; and
(b) detecting the polymeric HPR product, thereby detecting the target nucleic acid molecule.

2. The method of embodiment 1, wherein:
the HPR is an HCR,
the plurality of HPR monomers is a plurality of HCR monomers,
the first HPR monomer species is a first HCR monomer species,
the second HPR monomer species is a second HCR monomer species, and
the HPR product is an HCR product.

3. The method of embodiment 2, wherein the HCR monomers are provided as DNA hairpins.

4. The method of embodiment 3, wherein the HCR monomers provided as DNA hairpins do not form a polymeric HPR product in the absence of the initiator sequence.

5. The method of any of embodiments 2-4, wherein:
the plurality of HCR monomers consist of the first and second HCR monomer species;
the first HCR monomer species comprises a portion A1 and a portion A2;
the second HCR monomer species comprises a portion B1 and a portion B2;
A1 is capable of hybridizing to the initiator sequence;
A1 is capable of hybridizing to B2;
A2 is capable of hybridizing to B1; and
A2 and/or B2 is capable of hybridizing to the terminator sequence.

6. The method of any of embodiments 1-5, wherein a portion of an HPR monomer of the polymeric HPR product hybridizes to the terminator sequence.

7. The method of embodiment 6, wherein hybridization of the portion of the HPR monomer of the polymeric HPR product to the terminator sequence inhibits the portion of the HPR monomer from hybridizing to another HPR monomer.

8 The method of embodiment 6 or embodiment 7, wherein the portion of the HPR monomer of the polymeric HPR product hybridized to the terminator sequence has a higher melting temperature than the portion of the HPR monomer of the polymeric HPR product hybridized to another HPR monomer.

9. The method of any of embodiments 6-8, wherein the portion of the HPR monomer of the polymeric HPR product has greater complementarity to the terminator sequence than to another HPR monomer.

10. The method of any of embodiments 6-9, wherein hybridization of the portion of the HPR monomer of the polymeric HPR product to the terminator sequence terminates the HPR.

11. The method of any of embodiments 1-10, wherein the terminator sequence comprises deoxyribonucleic acid (DNA), ribonucleic acid (RNA) and/or locked nucleic acid (LNA).

12. The method of any of embodiments 1-11, wherein the terminator sequence is comprised by the target nucleic acid molecule.

13. The method of any of embodiments 1-11, wherein the terminator sequence is comprised by an adapter probe further comprising a hybridization region that hybridizes directly to the target nucleic acid molecule or to a branch probe or branch probe complex that hybridizes to the target nucleic acid molecule.

14. The method of embodiment 13, wherein the terminator sequence is on a 5′ and/or 3′ overhang of the adapter probe comprising the terminator sequence, wherein the overhang does not directly hybridize to the target nucleic acid molecule or to a branch probe or branch probe complex.

15. The method of embodiment 13 or embodiment 14, wherein the adapter probe comprising the terminator sequence comprises multiple copies of the terminator sequence.

16. The method of any of embodiments 1-15, wherein the initiator sequence is comprised by an adapter probe further comprising a hybridization region that hybridizes directly to the target nucleic acid molecule or to a branch probe or branch probe complex that hybridizes to the target nucleic acid molecule.

17. The method of embodiment 16, wherein the initiator sequence is on a 5′ and/or 3′ overhang of the adapter probe comprising the initiator sequence, wherein the overhang does not directly hybridize to the target nucleic acid molecule or to a branch probe or branch probe complex.

18. The method of embodiment 16 or embodiment 17, wherein the adapter probe comprising the initiator sequence comprises two or more separate nucleic acid molecules.

19. The method of any of embodiments 16-18, wherein the adapter probe comprising the initiator sequence is a split adapter probe, wherein:
the split adapter probe comprises a first adapter oligonucleotide comprising a first portion of the initiator sequence and a second adapter oligonucleotide comprising a second portion of the initiator sequence;
each adapter oligonucleotide further comprises a hybridization region, and the hybridization regions of the first and second adapter oligonucleotides hybridize to adjacent sequences on the target nucleic acid molecule, or to sequences separated by fewer than about 100 intervening nucleotides on the target nucleic acid molecule; and
upon hybridization of the first and second adapter oligonucleotides to the target nucleic acid molecule, the first and second portion of the initiator sequence are brought into proximity and hybridize to a monomer of the first HPR monomer species, thereby initiating an HPR reaction to generate a polymeric HPR product.

20. The method of any of embodiments 1-19, wherein the initiator sequence and terminator sequence are comprised by the same adapter probe.

21. The method of any of embodiments 1-19, wherein the initiator sequence and terminator sequence are comprised by different adapter probes.

22. The method of any of embodiments 1-21, wherein a plurality of adapter probes comprising the terminator sequence hybridize to the target nucleic acid or to a branch probe or branch probe complex that is hybridized to the target nucleic acid.

23. The method of embodiment 1-22, wherein at least 2, at least 5, at least 10, at least 15, or at least 20 adapter probes comprising the terminator sequence hybridize to a branch probe or branch probe complex that is hybridized to the target nucleic acid.

24. The method of any of embodiments 1-23, wherein a plurality of branch probes or branch probe complexes are each hybridized to (i) a plurality of adapter probes comprising the terminator sequence, and (ii) the target nucleic acid molecule.

25. The method of any of embodiments 22-24, wherein the branch probe complex comprises multiple amplifier probes bound to sites in a pre-amplifier probe.

26. The method of any of embodiments 22-25, wherein the method comprises contacting the biological sample with (i) the plurality of HPR monomers, (ii) one or more adapter probes, each independently comprising the initiator sequence and/or terminator sequence, and (iii) the branch probe or branch probe complex, simultaneously or sequentially in any order.

27. The method of any of embodiments 22-26, wherein the branch probe and/or branch probe complex is hybridized to the target nucleic acid molecule prior to contacting the sample with the plurality of HPR monomers and/or one or more adapter probes.

28. The method of any of embodiments 22-27, wherein the adapter probe comprising the terminator sequence is hybridized to the target nucleic acid or branch probe and/or branch probe complex prior to contacting the sample with the plurality of HPR monomers.

29. The method of any of embodiments 22-27, wherein the adapter probe comprising the terminator sequence is hybridized to the target nucleic acid or branch probe and/or branch probe complex subsequent to contacting the sample with the plurality of HPR monomers.

30. The method of any of embodiments 1-29, wherein the method further comprises contacting the sample with a terminator oligonucleotide that comprises the terminator sequence and does not comprise a separate sequence for hybridization directly or indirectly to the target nucleic acid molecule.

31. The method of any of embodiments 1-30, wherein the polymeric HPR product comprises fewer HPR monomers than a polymeric HPR product generated in a comparable HPR reaction carried out in the absence of the terminator sequence.

32. The method of any of embodiments 1-31, wherein at least a fraction of the HPR monomers are labeled with a detectable label.

33. The method of embodiment 32, wherein the detectable label is a fluorescent label.

34. The method of embodiment 32 or embodiment 33, wherein detecting the polymeric HPR product comprises detecting a signal generated from the detectable label.

35. The method of embodiment 34, wherein the signal generated from the detectable label has a reduced size and/or intensity in comparison to a signal generated from the detectable label in a comparable HPR reaction carried out in the absence of the terminator sequence.

36. The method of any of embodiments 1-35, wherein the target nucleic acid molecule is detected in situ in the biological sample.

37. The method of any of embodiments 1-36, wherein the target nucleic acid molecule is DNA.

38. The method of any of embodiments 1-36, wherein the target nucleic acid molecule is RNA.

39. The method of any of embodiments 1-38, wherein the biological sample is non-homogenized and optionally selected from the group consisting of a formalin-fixed, paraffin-embedded (FFPE) sample, a frozen tissue sample, and a fresh tissue sample.

40. The method of any of embodiments 1-39, wherein the biological sample is fixed.

41. The method of any of embodiments 1-39, wherein the biological sample is not fixed.

42. The method of any of embodiments 1-41, wherein the biological sample is permeabilized.

43. The method of any of embodiments 1-42, wherein the biological sample is embedded in a matrix, optionally wherein the matrix comprises a hydrogel.

44. The method of any of embodiments 1-43, wherein the biological sample is cleared, optionally wherein the clearing comprises contacting the biological sample with a proteinase.

45. The method of any of embodiments 1-44, wherein the biological sample is cross-linked.

46. The method of any of embodiments 1-45, wherein the biological sample is a tissue slice between about 1 μm and about 50 μm in thickness, optionally wherein the tissue slice is between about 5 μm and about 35 μm in thickness.

47. A composition, comprising a plurality of HPR monomers comprising a first HPR monomer species and a second HPR monomer species, wherein:
the plurality of HPR monomers are configured to (a) polymerize to generate a polymeric HPR product when a monomer of the first HPR monomer species hybridizes to an initiator sequence, and (b) terminate polymerization when a monomer of the HPR product hybridizes to a terminator sequence;
the first HPR monomer species and second HPR monomer species are each capable of hybridizing to another HPR monomer species; and
the initiator sequence and terminator sequence are each independently comprised by (i) a target nucleic acid molecule, or (ii) an adapter probe hybridized directly or indirectly to the target nucleic acid molecule.

48. The composition of embodiment 47, further comprising an adapter probe comprising the initiator sequence, and/or an adapter probe comprising the terminator sequence.

49. The composition of embodiment 47, further comprising an adapter probe comprising the initiator sequence and the terminator sequence.

50. The composition of any of embodiments 47-49, further comprising one or more of a branch probe or a branch probe complex, wherein each branch probe or branch probe complex is capable of hybridizing to (a) the target nucleic acid molecule, and (b) an adapter probe comprising the initiator and/or terminator sequence.

51. The composition of any of embodiments 47-50, further comprising the target nucleic acid.

52. A kit comprising the composition of any of embodiments 47-51.

### EXAMPLE

The following example is included for illustrative purposes only and is not intended to limit the scope of the present disclosure.

### Example 1: Detection of a Target Nucleic Acid in a Biological Sample Via Hybridization Chain Reaction (HCR) with Initiator and Terminator Sequences Associated with the Target Nucleic Acid

This Example discloses exemplary methods for in situ detection of target nucleic acids using hybridization-based polymerization reactions (HPRs), as exemplified by hybridization chain reaction (HCR). In some aspects, HCR is a sensitive method for detection and analysis of individual molecules in a biological sample, such as RNA transcripts. However, in general, HCR reactions proceed for as long as individual HCR monomers are present in the sample, which can lead to associated signals with intensities and/or sizes that are highly variable, and/or above an optimum range for detection.

In particular, this Example discloses exemplary methods for performing HCR using an initiator sequence and terminator sequence, which are each independently comprised by (i) a target nucleic acid molecule, or (ii) an adapter probe hybridized directly or indirectly to the target nucleic acid molecule. In some aspects, the terminator sequence is capable of terminating the HCR reaction, thereby affecting (e.g., limiting) the size and/or dispersity of (i.e., variance in number of monomers in) resulting polymeric HCR products. In some aspects, the terminator sequence is associated with the same target nucleic acid as the initiator sequence, such that proximity of the terminator sequence to the initiator sequence allows the HCR reaction to be predictably terminated by the terminator sequence.

A biological sample (e.g., fixed cells, a processed or cleared biological sample, a tissue sample, a sample embedded in a hydrogel, etc.), is contacted with an adapter probe comprising an initiator sequence and an adapter probe comprising a terminator sequence. In some examples, separate adapter probes comprise the initiator sequence and terminator sequence, respectively (e.g., as in FIG. 2D). In some examples, a single adapter probe comprises both the initiator sequence and the terminator sequence (e.g., as in FIG. 2E). In a separate control condition, the terminator sequence or the separate adapter probe comprising the terminator sequence is omitted. Each adapter probe is hybridized to the same target nucleic acid molecule. The sample is washed to remove unbound adapter probes.

Next, the sample is contacted with a first and second HCR monomer species, each species comprising a DNA hairpin HCR monomer, e.g. as shown in FIG. 4. In the current example, the first and/or second HCR monomer species comprises a fluorescent label. The first HCR monomer species comprises a self-complementary hairpin with an interior loop and an input domain with a toehold at the 5′ end. The second HCR monomer species comprises a self-complementary hairpin with an interior loop and an input domain with a toehold at the 3′ end. The input domain of the first HCR monomer species is capable of hybridizing to the initiator sequence, and the output domain of the second HCR monomer species is capable of hybridizing to the terminator sequence. When the first and second monomer species are added to the biological sample, the initiator sequence hybridizes to the input domain of a monomer of the first HCR monomer species, thereby exposing its output domain, which hybridizes to the input domain of a monomer of the second species, thereby exposing the output domain of the monomer of the second species, which hybridizes to the input domain of another monomer of the first HCR species, and so on. In this way, alternating hybridization events between monomers of the first and second HCR monomer species propagate the HCR reaction to form a growing polymeric HCR product. The HCR reaction is terminated (i.e. the polymeric HCR product stops growing) when the output domain of a monomer of the second HCR monomer species of the HCR product hybridizes to the terminator sequence, which inhibits the output domain from hybridizing to the input domain of another monomer. In the separate control condition lacking the terminator sequence, HCR termination is not controlled by a provided terminator sequence, and the HCR may proceed for as long as HCR monomers are present.

The sample is again washed to remove unbound probes (e.g., monomers), and the sample is imaged to detect a signal generated from the fluorescent label comprised by the first and/or second HCR monomer species comprised by the polymeric HCR product or reporter molecule hybridized to the polymer. In some aspects, the polymeric HCR product is an amplification product with repeated units of sequences that can be detected, incorporated by the plurality of monomers. One or more signals are detected in the biological sample. In some examples, the signals have smaller and/or lower intensities than the signals detected in the separate control condition lacking the terminator sequence. In some examples, the signals have more uniform sizes and/or intensities than the signals detected in the separate control condition lacking the terminator sequence.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
(a) contacting the biological sample with a plurality of HPR monomers comprising a first HPR monomer species and a second HPR monomer species, wherein binding of a monomer of the first HPR monomer species to an initiator sequence initiates an HPR reaction to generate an amplification product, wherein:
the first HPR monomer species and the second HPR monomer species are each capable of binding to another HPR monomer species;
at least one of the HPR monomer species is capable of binding to a terminator sequence; and
the initiator sequence and terminator sequence are each independently comprised by (i) a target nucleic acid molecule, or (ii) an adapter probe bound directly or indirectly to the target nucleic acid molecule; and
(b) detecting the amplification product, thereby detecting the target nucleic acid molecule.

2. The method of claim 1, wherein:
the HPR is an HCR,
the plurality of HPR monomers is a plurality of HCR monomers,
the first HPR monomer species is a first HCR monomer species,
the second HPR monomer species is a second HCR monomer species, and
the HPR product is an HCR product.

3. The method of claim 2, wherein the HCR monomers are provided as DNA hairpins, and wherein the HCR monomers do not form an amplification product in the absence of the initiator sequence.

4. (canceled)

5. The method of claim 2, wherein:
the plurality of HCR monomers consist of the first and second HCR monomer species;
the first HCR monomer species comprises a portion A1 and a portion A2;
the second HCR monomer species comprises a portion B1 and a portion B2;
A1 is capable of binding to the initiator sequence;
A1 is capable of binding to B2;
A2 is capable of binding to B1; and
A2 and/or B2 is capable of binding to the terminator sequence.

6. The method of claim 1, wherein a portion of an HPR monomer of the amplification product binds to the terminator sequence, and wherein binding of the portion of the HPR monomer of the amplification product to the terminator sequence terminates the HPR.

7. The method of claim 6, wherein binding of the portion of the HPR monomer of the amplification product to the terminator sequence inhibits the portion of the HPR monomer from binding to another HPR monomer.

8. The method of claim 7, wherein the portion of the HPR monomer of the amplification product bound to the terminator sequence has a higher melting temperature than the portion of the HPR monomer of the amplification product bound to another HPR monomer.

9-11. (canceled)

12. The method of claim 1, wherein the terminator sequence is comprised by the target nucleic acid molecule.

13. The method of claim 1, wherein the terminator sequence is comprised by an adapter probe further comprising a binding region that binds directly to the target nucleic acid molecule or to a branch probe or branch probe complex that binds to the target nucleic acid molecule, wherein the terminator sequence is on a 5′ and/or 3′ overhang of the adapter probe comprising the terminator sequence, and wherein the overhang does not directly bind to the target nucleic acid molecule or to a branch probe or branch probe complex.

14. (canceled)

15. The method of claim 13, wherein the adapter probe comprising the terminator sequence comprises multiple copies of the terminator sequence.

16-19. (canceled)

20. The method of claim 1, wherein the initiator sequence and terminator sequence are comprised by the same adapter probe.

21. The method of claim 1, wherein the initiator sequence and terminator sequence are comprised by different adapter probes.

22. The method of claim 1, wherein a plurality of adapter probes comprising the terminator sequence bind to the target nucleic acid or to a branch probe or branch probe complex that is bound to the target nucleic acid.

23-24. (canceled)

25. The method of claim 22, wherein the branch probe complex comprises multiple amplifier probes bound to sites in a pre-amplifier probe.

26. The method of claim 22, wherein the method comprises contacting the biological sample with (i) the plurality of HPR monomers, (ii) one or more adapter probes, each independently comprising the initiator sequence and/or terminator sequence, and (iii) the branch probe or branch probe complex, simultaneously or sequentially in any order.

27. The method of claim 22, wherein the branch probe and/or branch probe complex is bound to the target nucleic acid molecule prior to contacting the sample with the plurality of HPR monomers and/or one or more adapter probes.

28-29. (canceled)

30. The method of claim 1, wherein the method further comprises contacting the sample with a terminator oligonucleotide that comprises the terminator sequence and does not comprise a separate sequence for binding directly or indirectly to the target nucleic acid molecule.

31. The method of claim 1, wherein the amplification product comprises fewer HPR monomers than an amplification product generated in a comparable HPR reaction carried out in the absence of the terminator sequence.

32. The method of claim 1, wherein at least a fraction of the HPR monomers are labeled with a detectable label.

33-35. (canceled)

36. The method of claim 1, comprising imaging the biological sample using fluorescence microscopy, wherein the target nucleic acid molecule is detected in situ in the biological sample.

37-52. (canceled)
